(19) United States
(12) Patent Application Publication
(10) Pub. No.: US 2024/0415513 A1
(43) Pub. Date: Dec. 19, 2024

(54) LYMPHATIC ANASTOMOSIS DEVICES AND METHODS

(71) Applicant: Beth Israel Deaconess Medical Center, Inc., Boston, MA (US)

(72) Inventor: Dhruv Singhal, Chestnut Hill, MA (US)

(21) Appl. No.: 18/752,514

(22) Filed: Jun. 24, 2024

Related U.S. Application Data
(63) Continuation-in-part of application No. 17/132,946, filed on Dec. 23, 2020.
(63) Continuation-in-part of application No. 17/133,277, filed on Dec. 23, 2020.
(60) Provisional application No. 63/522,710, filed on Jun. 22, 2023.

(51) Int. Cl. A61B 17/11; A61B 17/00

(52) U.S. Cl. CPC A61B 17/11; A61B 2017/00022; A61B 2017/00367; A61B 2017/00477; A61B 2017/00862; A61B 2017/1132

(57) ABSTRACT

Preferred embodiments relate to devices and methods for performing a lymphovenous bypass procedure. A coupling element is secured to tissue connected to at least one lymphatic channel of a patient and a vein or artery of the patient. An end of the lymphatic channel that extends through the coupling element is inserted into an open end of the vein or artery to deliver lymph fluid from the lymphatic channel into the vein or artery.

### CROSS REFERENCE TO RELATED APPLICATION

This application claims priority to U.S. provisional application No. 63/522,710 filed on Jun. 22, 2023, and is also a continuation-in-part of International Application No. PCT/US2021/065126, filed Dec. 23, 2021 and is also a continuation-in-part of U.S. Non-Provisional application Ser. No. 17/133,277, filed Dec. 23, 2020, which is related to International Application No. PCT/US2020/038806, filed Jun. 19, 2020, which claims priority to U.S. Provisional Application No. 62/864,862, filed Jun. 21, 2019, the entire contents of each of the above applications is incorporated by reference herein. This application is also a continuation-in-part of application of U.S. patent application Ser. No. 17/132,946, filed Dec. 23, 2020, and a continuation-in-part of U.S. patent application Ser. No. 17/621,566, filed Dec. 21, 2021, the entire contents of these applications being incorporated herein by reference.

### BACKGROUND

The lymphatic system is a complex system of cellular tissue, vessels and organs that operates to carry excess fluids to the bloodstream and provides important functions to a body's immune system by removing pathogens from the circulatory system. The system includes small organs, or lymph nodes, that number around 500-600 in the human body. Lymphatic capillaries and vessels transport interstitial fluid typically through lymphatic ducts into the circulatory system. Interstitial fluid in the lymphatic system (“lymph”) can build up due to disease or injury. An excessive accumulation of this fluid is known as lymphedema.

Breast cancer-related lymphedema (BCRL) is one of the most significant survivorship issues in breast cancer management. Presently there is no cure for BCRL. Of 2.8 million breast cancer survivors in the United States, it is estimated that 1 in 5 suffers from BCRL. Patients presenting with BCRL often complain of tightness, heaviness, fatigue, and inability to fit into clothing secondary to swelling that is commonly experienced with this condition. In select cases, patients present with repeated episodes of rapidly spreading cellulitis of the affected extremity that can be life threatening if not treated expeditiously. The signs and symptoms of BCRL have been associated with a predilection towards anxiety, depression, and overall reduced quality of life. The most common risk factors for the development of BCRL are an axillary lymph node dissection, regional lymph node radiation (RLNR), and/or an elevated BMI (>30).

The standard treatment for BCRL has been physical therapy with manual lymphatic drainage, compression, local skin care, exercises, and pneumatic devices. Surgical management of chronic lymphedema can include lymphovenous bypass and lymph node transfer, however, these do not provide a definitive cure. The single greatest risk factor for developing BCRL is an axillary lymph node dissection (ALND). Lymphatic Microsurgical Preventative Healing Approach (LYMPHA) is a surgical procedure to reduce the risk of lymphedema in patients undergoing an ALND. LYMPHA has been used in patients undergoing ALND who developed lymphedema.

Note that a significant risk factor for the development for lymphedema is an ALND. In one study, 1 of 67 patients undergoing a sentinel lymph node biopsy developed lymphedema (1.5%). On the other hand, 4 of 10 patients who underwent an ALND alone developed lymphedema (40%). However, when LYMPHA was performed at the time of ALND, only 1 of 8 patients developed lymphedema (12.5%). Offering LYMPHA with ALND decreased the institutional rate of lymphedema from 40% to 12.5% in this study for example.

In the LYMPHA procedure, lymphatics draining the arm are identified and bypassed into an axillary vein tributary at the time of an axillary dissection. This technique has demonstrated a 5% lymphedema rate after axillary lymph node dissection (ALND) and LYMPHA over a four year period, for example. Historical rates of lymphedema after ALND are highly variable however, often indicated to be between 20-40% and have been reported as high as 77%.

One challenge of the LYMPHA procedure is visualizing healthy cut lymphatics lateral to the level 1 lymph nodes after an ALND. A technique for identifying these lymphatics can use an injection of blue dye into the ipsilateral proximal upper arm to visualize location. Although most LYMPHA procedures have been performed in the axillary bed, note that other lymph node dissection locations including the neck, chest, abdomen and groin carry a risk of lymphedema development and a bypass can reduce the risk of lymphedema development at these sites and associated extremities. Notwithstanding the improvements in the treatment of lymphedema with the above referenced procedures, further improvements are needed in this procedure to improve the treatment of this condition.

### SUMMARY

The present invention relates to a device for coupling one or more lymphatic channels to the vascular system. Of significant note, other previously described lymphatic and vascular anastomotic devices require vessels of similar caliber to be connected in an end-to-end manner or an end to side manner for a size discrepancy. Preferred embodiments as described herein enable the intussusception of one or more lymphatic channels of significantly different size into a single vein, for example. Consequently, these preferred embodiments of coupling devices facilitate the LYMPHA procedure by improving the speed of the procedure, improving the stability of the resulting anastomosis, and can serve to couple a single lymphatic channel, or plurality of lymphatic channels, into a single vascular channel such as a vein or artery.

The procedure can utilize the fat tissue associated with grouping of one, two or more lymphatic channels to assist in connecting the lymphatic channels to a first coupling element of the device. Prior to beginning the procedure, the lymphatic system in a region of interest can be evaluated by visualization techniques. Dyes may be injected for microscopic imaging and lymphatic mapping to identify a specific region of interest that would serve to drain fluid from an affected region such as an arm of a patient. The surgeon begins the procedure by accessing the site by incision to expose lymphatic channels and one or more veins that can be used, and identifying one of more lymphatic channels to be coupled into a selected vein. Visualization of the implanted device can be improved with fluoroscopic markers attached to, or imbedded within, or positioned on one or more regions of the device. Visualization of lymph flow after implantation of the connector to couple the lymph channels into the vein, or coupling to one or more tributaries of the vein, can be used to monitor the viability of the lymph flow after closure of the surgical wound.

A preferred embodiment of a coupling device can comprise a coupling element having an aperture extending through the coupling device to receive a vein or artery that is attached to one or more first tissue grasping elements on one side so as to provide an exposed open end of the vein or artery. The open end of the artery is sized to receive one or more lymphatic channels that are inserted to a depth within the vein or artery. The lymphatic channels can be stabilized within the vein or artery by attaching tissue that adheres to the lymphatic channels to one or more second tissue grasping elements on a second side of the device. In preferred embodiments, the device can have rounded exterior surfaces so as not to abrade surrounding tissue during or after the procedure by which the device is implanted into the body to provide lymphovenous bypass for the treatment of lymphedema. This embodiment provides for the insertion of the vein or artery into the aperture, and after coupling the vein or artery to the first side of the device, inserting the lymphatic channels into the open end of the artery and then moving the tissue in which the lymphatic tissue is positioned over the outer surface of the device and coupling the tissue to the second side of the device. This embodiment thus provides a unitary device for grasping both the vein or artery and for coupling to the lymphatic channels to stabilize them in the vein or artery and for implantation of the coupling device into the patient as described generally herein. For embodiments of the tissue grasping elements on one or both sides of the device in which it is necessary to further cover the tissue grasping elements, one or more caps can be used to enclose any exposed features of the tissue grasping elements to prevent further abrasion of the surrounding tissue after implantation.

A specific coupling device may be selected based on the number and size of the lymphatic channels and the vein to which they are to be positioned. The device can be fabricated by standard molding and assembly techniques using biocompatible materials such as synthetic polymers or silicone, for example. These may have different sizes and shapes depending on the particular site for implantation. A vein can be attached to a second coupling element that can include an aperture or opening from 1.0 mm to 3.0 mm in diameter, for example. The first and second coupling elements can be shaped as rings with the lymphatic channels connected to extend through the central opening of the first ring and a vein connected to the second ring, the first ring being attached to the second ring such that one or more lymphatic channels extend into the single vein, i.e., the lymphatic channels are intussuscepted into the vein.

In a further embodiment, a connector device can be used to align and connect the first ring to the second ring. The connector device can include one or more cone shaped elements, for example, with a connector channel through which the lymphatic channels can extend through the second ring opening into the vein, that is, the lymphatic channels are intussusepted, or telescoped into the vein. The cone shaped element(s) can include a shaped surface or surfaces that extend from a larger diameter portion to a smaller diameter portion that extends around the opening of a cavity through which the lymphatic vessels slide into the vein. The cavity can comprise a tubular channel through which the vein is inserted with the wall of the vein being engaged by pins or tissue anchors. The lymphatic vessels and capillaries are embedded within supporting tissue that generally surrounds the vessels. Note that when a lymph node has been removed to address a patient's medical condition, the lymphatic vessels that were connected to that lymph node have been cut and will typically continue to pass lymph fluid that will flow through the cut ends and into the surrounding tissue. The surgeon can expose the ends of the lymphatic vessels that have been cut wherein the ends extend a distance from that portion of the supporting tissue that is attached to the pins or tissue anchors that are sized and shaped to grasp the supporting tissue. The supporting tissue can comprise a fibrous connective tissue that can be manually grasped by the surgeon using forceps or tweezers, for example. These can include grasping instruments having a small tip size to grasp the lymphatic vessels that can have a diameter in a range of 0.1 mm to 1.0 mm, or larger, that are positioned within, or in proximity to, an exposed end of a vein into which the lymph fluid is to be delivered. There are frequently three or four lymphatic vessels in a region about a removed lymph node that can be grasped using the surrounding adipose tissue that supports these lymphatic vessels. The lymphatic vessels are spaced apart, with the surgeon frequently selecting a group of lymphatic vessels for the anastomosis that are spaced sufficiently close that they will fit into the diameter of the vein. Lymph nodes can vary in size depending on the age and medical condition of the patient, but will frequently have a size in a range of 4 mm to 2 cm along the long axis of the lymph node. The size of the present device can vary depending on the size of the vein to be attached to the device, but can also be in a range of 4 mm to 2 cm in diameter. By mounting the supporting tissue onto pins or anchors, this mounting movement tends to cause the cut ends of the lymphatic vessels to extend further from the surrounding tissue and thereby be readily placed into the exposed end of a vein where the end of the vein is mounted onto the pins or anchors. The open end of the vein can be slightly enlarged. The lymphatic vessels can have varying lengths extending from the supporting tissue and thus may be placed into the vein at different insertion lengths or there may be ends of lymphatic vessels that reside outside the end of the vein that nevertheless continue to deliver lymph fluid into the vein. Some of the lymphatic vessels will have higher flow rates so that even those residing outside of the vein after implant of the device and closer of the surgical wound will have sufficient proximity to the vein to preserve flow. A tissue flow channel may form that extends from the end of a high flow lymphatic vessel into the vein.

The first coupling element can have tissue grasping elements such as pins, prongs or tissue anchors that grasp the fatty tissue surrounding the lymphatic channels. Thus, the lymphatic channels extend within channel supporting tissue that can be attached to the first coupling element without impairing lymphatic channel function thereby enabling transport of lymph into the vein such that swelling is reduced. The pins, posts, prongs or tissue anchors can extend through the fatty tissue to engage receiving features on the second coupling element. For embodiments utilizing a connector element between a first ring and a second ring, for example, the pins, prongs or tissue anchors may engage the tissue, and may also engage the connector element.

Surgical tools can be used to grasp the tissue to position it relative to the pins, prongs or tissue anchors to thereby attach the tissue to the anastomosis device. A clamping device can be used to temporarily hold the coupling elements of the device in position to facilitate the attachment of tissue to each element, alignment of the coupling elements and connecting the components together, as needed.

Medical personnel can perform procedures as described herein by first evaluating a patient's condition in which swelling has occurred, or is likely to occur. Visualization techniques as described herein can be used to map those regions of the lymphatic system to select one or more regions thereof that will reduce or eliminate swelling by implantation of one or more devices as described herein. As preferably at least 2, 3, 4, 5 or more lymphatic channels can be fluidly coupled into a single vein, a significant amount of lymph can be removed into a single vein using a single device. After mapping and selection, one or more devices are implanted as described herein.

Further embodiments employ a device in which the lymphatic channels are grasped individually or collectively and placed within the vein at a selected depth. This can be performed manually or by using a robotic device. For example, forceps and/or a loop of suture material can be used to grasp one or more channels and used to place them into the vein attached to the tube or ring. The suture material can be temporarily attached to the tube before biodegrading after closure of the wound over a time period in which the channel tissue and vein tissue heal so as to permanently connect the channels to the vein. A biocompatible adhesive can also be used to attach tissues to the tube, for example. The tube can have surface elements or grooves allowing it to be held by the surgeon for attachment of the vein inserted on one side and connected to pins or prongs as described herein and the channels inserted into the vein at the opposite end of the tube. For robotic surgery, a plurality of controlled arms with manipulating elements can be used isolate and grasp the vein and attach the exposed end to the first coupling element. The controlled arms can also operate to attach the tissue, such as visceral adipose tissue containing the lymphatic vessels, to the second coupling element as described herein. The robotic arms can then be controlled by the surgeon to grasp the first and second elements, bring them into alignment and attached them together as to fluidly couple the lymphatic channels into the vein. The device can then be positioned within the wound opening and the wound sutured so as to close the wound.

In further embodiments, the one or more lymphatic vessels can be fluidly coupled to a vein by attaching the vein and the vessels to a single unitary coupling element. The unitary coupling element can comprise a generally cylindrical body wherein the vein is coupled to a first end of the cylindrical body and the lymphatic vessels to a second end of the cylindrical body. Embodiments can further include a more rounded or oval shape. The exterior surface can have slots or grooves that enable a user to more readily grasp the implant device manually or with grasping surgical instruments such as forceps or tweezers. The unitary coupling element will have a first opening to receive the vein at the first end with a first group of pins or anchors positioned around the inside portion of the first opening to secure the open end of the vein within the implant. The second end can have a wider opening than the vein insertion opening to provide for the insertion of the supporting tissue which typically has a larger diameter than the vein as it must incorporate all the spaced apart lymphatic vessels to be inserted into the vein. The sidewall of the implant device that extends around the periphery of the second opening can have sidewall openings or windows through which the surgeon can insert a surgical grasping tool such as forceps, for example, that grasp different regions of the supporting tissue for placement onto the a second group of pins or anchors that generally extend in an opposite direction from the first group of pins or anchors that secure the open end of the vein. The pins or anchors can extend along the longitudinal axis of the implant device, or they can extend radially or at some angle to the longitudinal axis between 1 and 45 degrees, for example, so as provide a more secure attachment.

Note a unitary body tissue coupling device generally refers to a coupling device having first tissue grasping elements on a first side and second tissue grasping elements on a second and wherein no assembly is required during surgical implantation. Thus, for example, the implementations of the coupler device in which there are at least two coupler components that each have tissue grasping elements and that must be assembled during use to provide for the coupling of fluid channels do not constitute a unitary body tissue coupling device.

### BRIEF DESCRIPTION OF THE DRAWINGS

FIG. 1 illustrates an embodiment of a coupling device using a pair of rings to align and connect lymphatic channels to a vein or artery.

FIG. 2A illustrates an embodiment including a connector element to attach a pair of rings together that couple one or more lymphatic channels to a vein.

FIG. 2B illustrates a further embodiment including a central tube connected to a ring and an external sheath can be attached to enclose the coupling elements.

FIG. 2C illustrates a further mechanism for connecting a first coupling element to a second coupling element.

FIG. 2D illustrates a perspective view of a first end of an implant for a lymphovenous bypass procedure.

FIG. 2E illustrates a further perspective view of a second end of the implant shown in FIG. 2D.

FIG. 3 illustrates an axillary anatomic region after dissection of level 1 and 2 lymph nodes where lymphatic channels glow from an FITC injection, described hereinafter and the bypass procedure has been performed.

FIG. 4 schematically illustrates the steps of a surgical procedure to perform a bypass surgical procedure in accordance with embodiments of the invention.

FIG. 5 schematically illustrates the lymphatic channel system of the human body wherein a bypass procedure in accordance with the invention can be performed at different locations to reduce lymphedema.

FIG. 6 illustrates a flowchart of lymphedema protocols used in conjunction with surgical procedures described herein.

FIG. 7 illustrates a robotic control system having computerized control of arms with manipulators such that a surgeon can perform procedures in accordance with preferred embodiments hereof.

FIG. 8 illustrates a process flow diagram for performing a robotically controlled surgical process in accordance with preferred embodiments.

FIG. 9 illustrates a sensor mounted to a connector device for measuring flow of lymph fluid into the vein.

FIG. 10 illustrates a valve device for controlling fluid pressure at a junction in the vein at which lymph fluid enters into the vein.

FIG. 11 illustrates a further embodiment of a coupling device and securing elements in accordance with certain embodiments.

FIG. 12 illustrates a front view of the coupling device of FIG. 11.

FIG. 13 illustrates a left side cross-sectional view of the coupling device taken at the line indicated in FIG. 12.

FIG. 14 illustrates a rear view of the coupling device of FIG. 11.

FIG. 15 illustrates a front view of the coupling device of FIG. 11.

FIG. 16 illustrates a side view of a further embodiment of a first coupling element according to certain embodiments described herein.

FIG. 17 illustrates a top view of the first coupling element of FIG. 16.

FIG. 18 illustrates a side perspective view of the first coupling element of FIG. 16a.

FIG. 19 illustrates a side view of the first coupling element.

FIG. 20 illustrates a top view of the first coupling element.

FIG. 21 illustrates an embodiment of a pin compatible with first and second coupling elements described herein.

FIG. 22A illustrates a perspective view of an embodiment of the second coupling element including pins.

FIG. 22B illustrates a perspective view of an interior volume of the coupling device of several embodiments described herein.

FIG. 23 illustrates a rear perspective view of a further embodiment of a first coupling element including pins.

FIG. 24A illustrates a front perspective view of a further embodiment of a coupling device.

FIG. 24B illustrates a top perspective view of a further embodiment of a coupling device.

FIG. 24C illustrates a bottom view of a further embodiment of a coupling device.

FIG. 25A illustrates a bottom view of a first coupling element of a further embodiment of a coupling device in accordance with certain embodiments.

FIG. 25B illustrates a top view of the first coupling element of FIG. 25A.

FIG. 25C illustrates a top perspective view of the first coupling element of FIG. 25A.

FIG. 25D illustrates a front perspective view of the first coupling element of FIG. 25A.

FIG. 25E illustrates a side perspective view of the first coupling element of FIG. 25A.

FIG. 25F illustrates a side view of the first coupling element of FIG. 25A.

FIG. 25G illustrates a perspective view of a first coupling element with a breakable handle in accordance with some embodiments.

FIG. 25H illustrates a perspective view of a first coupling element without handle in accordance with some embodiments.

FIG. 25I illustrates a perspective view of a first coupling element including recesses for forceps in accordance with some embodiments.

FIG. 26A illustrates a perspective view of the first coupling element of FIG. 25A and a second coupling element.

FIG. 26B illustrates a side view of the first coupling element of FIG. 25A and the second coupling element of FIG. 26A.

FIG. 27A illustrates a perspective view of the first coupling element of FIG. 25A and a second coupling element.

FIG. 27B illustrates a side view of the first coupling element of FIG. 25A and the second coupling element.

FIG. 28A illustrates a perspective view of the first coupling element of FIG. 25A and a holder for attaching a second coupling element to the first coupling element.

FIG. 28B illustrates a side view of the first coupling element of FIG. 25A and the holder.

FIG. 29A illustrates a perspective view of the first coupling element of FIG. 25A attached to a second coupling element by the holder of FIG. 28A.

FIG. 29B illustrates a side view of the first coupling element of FIG. 25A attached to a second coupling element by the holder of FIG. 28A.

FIG. 30 depicts an exemplary method of performing a lymphovenous bypass surgical procedure.

FIGS. 31A and 31B illustrates an exemplary perspective view of a grasping tool inserted through a coupler element to grasp lymphatic channels in performing lymphatic bypass surgery and an enlarged view of the distal grasping elements of the grasping tool.

FIGS. 32A and 32B is a process flow diagram for performing lymphatic bypass surgery with a unitary coupler as described herein.

### DETAILED DESCRIPTION OF THE DRAWINGS

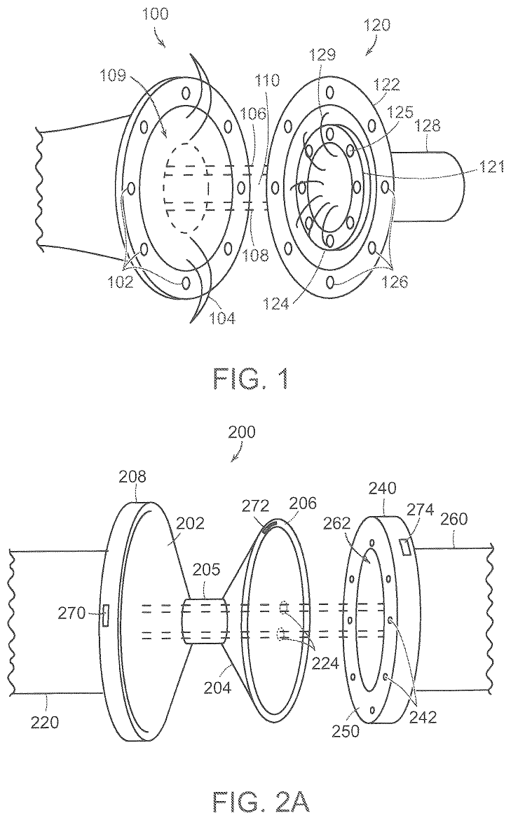

Preferred embodiments of the invention utilize a device for coupling one or more lymphatic channels to the vein of a patient's circulatory system. Shown in FIG. 1 is an embodiment of a coupling device in which a first coupling element 100 comprises a ring with a central opening 109 through which the fatty tissue 104 surrounding the lymphatic channels 106, 108 is drawn over pins, prongs or tissue anchors 102 that face inwards from the inner ring surface towards second coupling element 120. Unlike vascular anastomeric couplers used to connect the ends of two blood vessels, the present invention enables the insertion of one or more lymphatic channels into (i.e, an intussusception) an open end of a blood vessel or vein as there is typically a mismatch in size, one lymphatic channel being substantially smaller than the size of the vein into which it is inserted. Thus, one, two or more lymphatic vessels with channels for flow of lymph fluid can be inserted into a single vein 128, depending on the sizes thereof. Note that blood vessels cannot be inserted into one another as clotting can result in anastomosis failure. Lymph fluid, on the other hand, does not clot. The insertion of lymphatic channel tissue into a blood vessel does not induce such clotting and allows flow of lymph into the vessel without obstruction.

The components in FIG. 1 can comprise a rigid or semi-rigid compliant, elastic biocompatible material with generally smooth surface features except for the pins, prongs or tissue anchors that are configured to penetrate and grasp tissue. In selected embodiments, the components provide a sutureless tissue connector, however sutures can be used to augment implantation of the device in some embodiments.

Components of the device can be made using biocompatible materials such as silicone, polyurethane, polytetrafluoroethylene (PTFE), polyesther, polyethylene, polyamide, polyetheretherketone (PEEK), polypropylene, Mylar, Kevlar, polyisoprene, polyolefin, or combinations thereof.

The first coupling element can comprise a ring having a larger opening to accommodate a thickness of fatty tissue, such as visceral adipose tissue (containing lymphatic vessels with channels extending through the vessels to transport lymph fluid), to extend therethrough and surround the lymphatic tissue, which consequently does not contact the connector surfaces. Note that ring elements can have other shapes, such as an oval cross-section, or some other shape suitable for a specific anatomical placement in the patient. The outer surface is preferably smooth to avoid abrading adjacent tissue. Lymphatic vessels are thin walled tubular shaped tissue structures that are lined with endothelial cells and comprise smooth muscle that is connected to surrounding tissue with adventitia. Lymphatic capillaries are smaller, without the muscle and adventitia, and range in diameter from 15-75 microns. The larger lymphatic vessels have valves spaced along their length with fluid movement provided by peristalsis to move lymph fluid through the vessel under fluid pressure. Lymphatic collecting vessels have a diameter in a range of 100-800 microns or larger. A vein of the vascular system can have a diameter of 1 mm or more and can be selected to receive two or more lymphatic channels for each vein selected. Generally a vein will have a diameter in the range of 2-4 mm that will be coupled to lymphatic vessels with supporting tissue that fits within a device aperture that is larger than the diameter of the aperture holding the vein. The coupling elements can have a diameter in a range of 1-15 mm and can have matching diameters in embodiments including two coupling elements. The present devices and methods can also be used to couple to one or more smaller tributary veins that feed into a larger vein. The inner surface of the central opening in an inner ring can be large enough to allow passage of the vein through the central opening such that the exposed end of the vein can be attached to the second connector. Thus, the second connector 120 can have the inner ring 124, with pins, prongs, or tissue anchors 125 that engage the tissue of the vein 129 that folds over the pins 125. The outer ring 122 has pin receiving regions 126 that receive and engage the ends of pins 102, for example, that protrude above the ring surface at an elevation sufficient to at least engage the tissue. Region 126 can be configured to snap together with at least some of the protruding elements or pins 102 from surface of ring 100 to provide a snap connector. A latching mechanism or other connector can be used to secure the coupling elements together. These features are illustrated in one or more of the figures described herein.

Note that ring element 124 can be elevated above the surface of ring 122 by one or more millimeters. Peripheral wall 121 can thus have a height of at least 1 mm. This can provide for the insertion of lymphatic channels 106 to be a depth of at least 1 mm into the vein 128, for example. Thus, the relative dimensions of the coupling elements can define a depth of insertion.

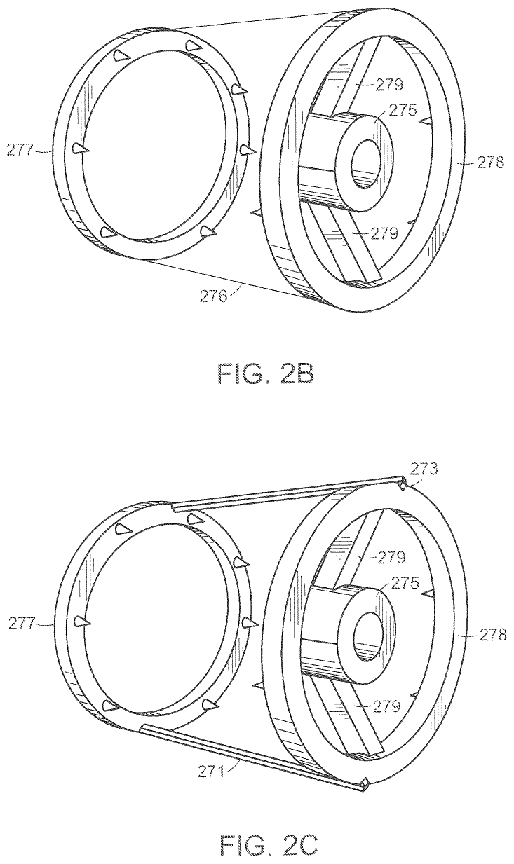

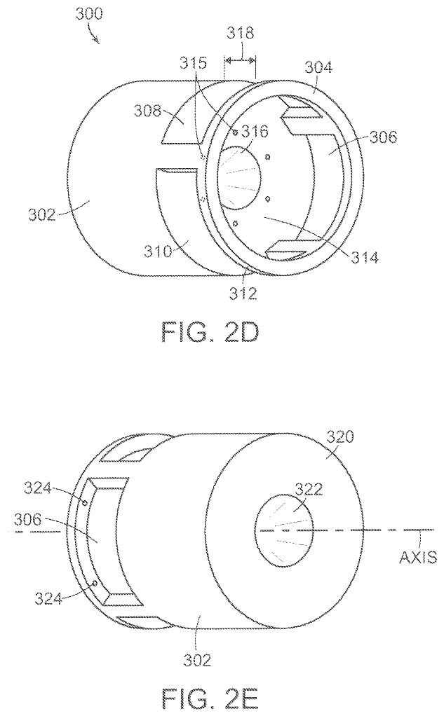

Shown in FIG. 2A is a coupler 200 having a first ring 208 that receives and anchors the vein 220 as described previously, however this mates to a first cone 202 shaped element having an open end that gradually narrows in diameter to a first end to an opening of a small diameter tube 205 where the lymphatic channels 224 are received from second cone 204 that narrows to the second end of tube 205. The wide end of cone 206 is sized and shaped to attach to an inward facing surface of first ring 240, that has pins, prongs or tissue anchors 242 that engage and secure adipose tissue with the lymphatic vessels 224 to the ring 240 wherein the tissue 260 surrounding the lymphatics is folded over the anchors 242 on surface 250, for example. The lymphatic vessels thus enter the open end of cone 206 and pass through a narrow opening into the tube 205 and into the vein. Note that the embodiment of FIG. 1 can employ a single cone that guides entry of the lymphatic vessels from the open end of the cone through a narrow opening of the cone and into the vein to define the junction at which lymph fluid enters the vein. Returning to FIG. 2A, the second ring 208 can include a first fluoroscopic marker 270, the cone 206 can include a second fluoroscopic marker 272, and second ring 240 can include a third fluoroscopic marker 274, for example, to illustrate the use of markers on the various embodiments described herein. Shown in FIG. 2B is a further embodiment in which first ring 277 can be connected to a second ring 278. Tube 275 is connected to ring 278 by a plurality of arms or connecting elements 279. The tube 275 has an internal cavity in which the vein can be inserted and attached to the tube as described herein. In preferred embodiments, the first and second rings can align upon a common axis when they are connected together. The pins or anchors can be arranged symmetrically about the common longitudinal axis. The pins or anchors for the vein can project in a first direction parallel to the common axis, whereas the pins or anchors for the connective supporting tissue can extend parallel to the common axis but in a second opposite direction.

The embodiments described herein can be encapsulated within an outer sheath 276 extending around the rings that are aligned along a common axis upon being connected together. The first coupling element or ring can be connected to the second coupling element with one or more connector elements. As described herein, connector elements such as pins, posts or prongs can be used. As shown in FIG. 2C, a plurality of prongs 271, 273 can extend from the first ring to the second ring where inwardly facing protrusions or ridges grasp the outer edge of the second ring. The outer sheath or surfaces of the devices provide a smooth outer surface. Certain elements of the device can be flexible so as to move with the surrounding tissue of the patient. One or more elements of the device can comprise bioabsorbable materials. Selected surfaces may be porous so as to accommodate ingrowth and adhesion to tissue adjacent the device to stabilize the device within the tissue matrix. The length of the tube 275 (or tube 205) can be used to indicate to the user that the length of the lymphatic channels that extend into the vein are sufficiently long to prevent the lymphatic channel from becoming dislodged from the anastomosis. There is a junction region, preferably within the device housing, where the lymphatic vessels deliver lymph fluid into the vein. Note that the first and second coupling elements can optionally be connected on one side so that the user can simply rotate the two components relative to each other around a pivot axis to align and connect the elements while the channels are inserted into the vein. In a further embodiment an outer sheath 276 can be attached to the device that extends around the circumference of the device and thereby enclose the device. The sheath 276 can also comprise portions extending peripherally from each ring that connect together by a sheath connector.

In a further embodiment, the coupling device can be fabricated as a single unitary piece that has a tubular portion to receive the vein though a first end such that the wall of the vein can be grasped by pins or anchors on a second end of the tubular portion. The second end of the device can have a larger opening that receives the supporting tissue containing the lymphatic vessels that are inserted into the vein. As shown in front view in FIG. 2D and in rear view in FIG. 2E, a coupling device 300 according to some embodiments described herein can have a body formed of a single, unitary piece, which can be fabricated using standard molding techniques, or by using three dimensional (3D) printing methods. In this embodiment, the coupling device 300 is defined by a cylindrical wall 302 that at least partially surrounds different portions of the coupling device 300. The cylindrical wall 302 can have sidewall openings 306, 308, 310 adjacent to an outer ring sidewall 312 in some embodiments. The sidewall openings 306, 308, 310 are sized to allow a user to pass a forceps or other tool first through one of the sidewall openings and then through the ring opening and past a bottom ring surface 304. The forceps can be used to grasp a tissue (such as adipose tissue surrounding lymphatic channels) and pull it through the ring opening and onto tissue grasping elements 324, such as pins anchors, at each opening 306, 308, 310. The pins may be inserted into pin securing areas or may be integrally formed with the coupling device 300, for example. In some embodiments, the coupling device 300 can have a center aperture 316 that extends from an inner bottom surface 314 to a top surface 320 of the coupling element 300. A vein can extend through the center aperture 316 from the top surface 320 and attached to tissue grasping elements 315 such as pins that may be inserted into pin securing areas or may be integrally formed with the coupling device 300. The tissue grasping elements 315 can protrude from the inner bottom surface 314, which is set at a depth 318 below the ring element. In some embodiments, the adipose tissue can be stretched onto the pins 324, which may cause extension of lymphatic vessels within the adipose tissue so that they will preferably extend into the vein secured at pins 315. The bottom ring surface opening has a larger size or diameter than the center aperture 316 as it must accommodate insertion of a larger volume of supporting tissue that contains the lymphatic channels to be fluidly coupled to the vein.

At the top surface 320, the surface around the center aperture 316 can be a cone-shaped surface 322. The cone-shaped surface 322 can help direct insertion of tissue by guiding the tissue into the center aperture 316 as force is applied to the tissue. In addition, the cone-shaped surface 322 can reduce abrasion on tissue (e.g., vein) that has been inserted as it does not have sharp corners or edges.

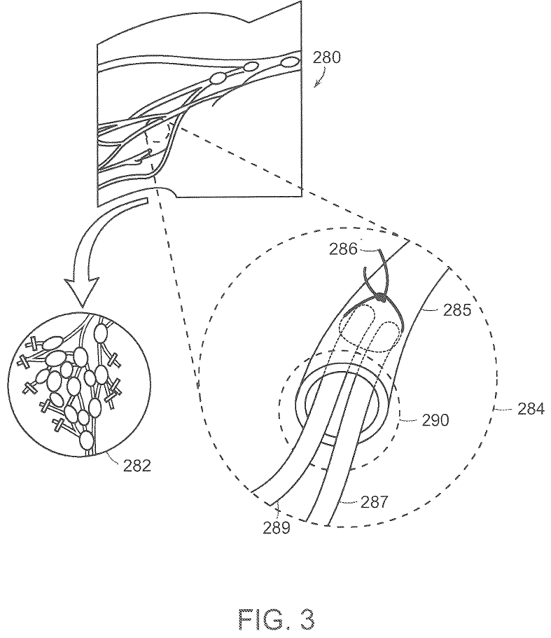

FIG. 3 shows an enlarged view 284 of the axillary location 280 that includes lymph nodes 282 and a vein 285, that has received a pair of lymphatic vessels 287, 289. Unlike prior procedures which used a suture 286 to secure the channels to the vein 285, the present invention uses a coupler 290 at the junction of the channels 287, 289 entering the vein.

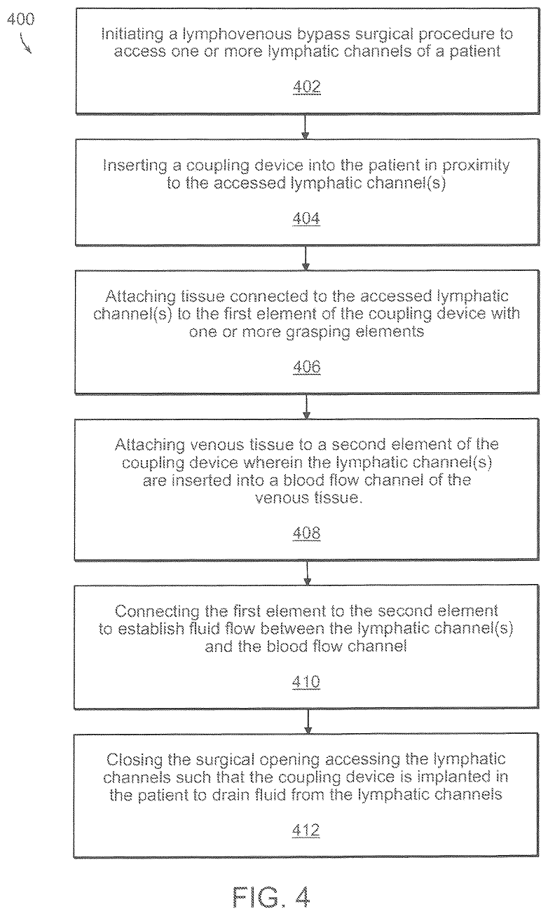

Shown schematically in FIG. 4 is a method 400 of performing a surgical procedure wherein, for example, a surgeon can perform an incision through the skin to access 402 tissue that includes one or more lymphatic channels. A coupling device is positioned into the patient 404 where a first coupling element is attached 406 to one or more lymphatic channels and a second coupling element is attached 408 to a vein. The first coupling element is connected 410 to a second coupling element such that the one or more lymphatic channels are positioned in the exposed vein opening to a depth such that lymph from the lymphatic channels can flow into the vein. The surgeon then closes 412 the surgical opening such that the coupling device is implanted in the patient. Alternatively, the coupling device can comprise a single tube or ring having pins or tissue anchors on one end to connect to a vein inserted into one open end of the tube. The wall tissue of the vein is placed onto the pins or tissue anchor elements which penetrate the wall tissue to hold the vein in place relative to the device. The lymphatic channels can be inserted through the tube opening at the opposite end and into the vein positioned at least partially within the tube. The tube may have internal features that permit insertion in one end but inhibit removal of the vein. Thus, an inner wall of the tube can have a frictional surface with teeth, pins, or other features directed in one direction to inhibit movement of the vein in the tube. The tube can have external features allowing a loop of material grasping the channels to be attached to the tube.

Dyes can be used to aid in visualization and mapping of the lymphatic system. Fluorescein isothiocyanate (FITC), for example, is excited in the visible spectrum and routinely used in the operating room. Neurosurgeons inject this dye intravenously and utilize microscopes equipped with filter technology to visualize tumors while maintaining life-like color of the surrounding tissues allowing for simultaneous magnification and tissue dissection. This is important for the lymphatic surgeon. Thus FITC can be used in the operating room for lymphatic mapping. Note, further that FITC has been utilized to perform a lymphovenous bypass (LVB) in the superficial tissues of the arm in a patient with chronic lymphedema. FITC is a safe and highly effective dye for lymphatic mapping and dissection in open surgical fields such as in the LYMPHA procedure.

Lymphedema repository data on all breast cancer patients that underwent the LYMPHA procedure included demographic information (age, body mass index [BMI]) and peri-operative data have been obtained (number of lymphatic channels visualized and bypassed, distance of channels from axillary vein, name of targeted vein, and adverse events).

In an exemplary procedure (see Spiguel et al. “Fluorescein Isothiocynate: A Novel Application for Lymphatic Surgery”, Annals of Plastic Surgery, Volume 78 (2017), the entire contents of which is incorporated herein by reference), prior to the ALND, 2 cc of a modified 2% fluorescein solution are injected intradermally and along the muscle fascia of the ipsilateral upper arm, for example. The solution can be modified from the stock AK-FLUOR 10% (Akorn Inc., Lake Forest, IL) solution by diluting 2 cc with 7.5 cc of normal saline and 0.5 cc of AlbuRx5 (CSL Behring Inc., King of Prussia, PA). The ALND is performed with attention to preserving a superficial accessory vein tributary which longitudinally traverses the level I lymph nodes. The superior dissection of the level I axillary contents along the axillary vein is performed with identification of the accessory vein tributary which is typically found anterior to the thoracodorsal neurovascular bundle. The vein is then dissected free from the level I axillary contents and clipped distally to provide maximal length. Completion of the level I and II ALND is then performed.

Following completion of the axillary dissection, for example, a Pentero 900D Microscope (Carl Zeiss Inc., Germany) equipped with the YELLOW 560 package, can be utilized to identify and map the divided lymphatic channels draining the arm. The harvested vein is prepared per standard microsurgical techniques. Utilizing existing techniques, a surgeon, using 9-0 nylon suture, places a “U” stitch to capture the anterior wall of the vein and parachute in the lymphatic channels chosen for bypass. 10-0 nylon can then be utilized to suture the wall of the vein to the perilymphatic tissue. Channels not bypassed are clipped. Lymphatic flow filling the vein can be visualized with the filter activated one hour after anastomosis.

However, in accordance with selected embodiments, the surgeon, instead of suturing, will attached the perilymphatic tissue to a first connecting element and attach the vein to a second connecting element, insert the exposed ends of the lymphatic channels into the opening at the vein and connect these components to securely complete the anastomosis or intussusception of lymphatic channels into the vein.

As noted in the study by Spiguel, et al, thirteen patients underwent LYMPHA with intra-operative FITC lymphatic imaging from March to September 2015. Average patient age was 50years with a mean BMI of 28. On average, 3.4 divided lymphatic channels (range 1-8) were identified at an average distance of 2.72 cm (range 0.25-5 cm) caudal to the axillary vein. 1.7 channels were bypassed per patient (0-4). Anastomoses were performed to the accessory branch of the axillary vein and or to a lateral branch. LYMPHA added an average of 67 minutes (45-120 minutes) to the oncologic procedure in these examples.

Thus, FITC is a safe and effective dye for the LYMPHA technique. In comparison to ICG and blue dye, FITC has many advantages. FITC does not permanently stain surrounding tissues, as opposed to ICG and blue dyes, which facilitates dissection of the lymphatic channels. The primary advantage of FITC over ICG in lymphatic surgery, for example, is the ability to allow for simultaneous visualization and dissection of lymphatic channels as FITC is excited in the visible spectrum making it a dye to be used in open surgical fields.

Diagnosed breast cancer patients can have a lymphedema evaluation pre-operatively. Each evaluation, pre-operatively and post-operatively, can include three components: (1) evaluation by a certified lymphedema therapist for signs and symptoms of BCRL, (2) circumferential measurements, and (3) bioimpedance spectroscopy. Lymphedema can be defined as having signs/symptoms of BCRL and one positive objective measure and can be transient or extend beyond 6 months, for example. Demographics (age, BMI, prior radiation or chemotherapy), cancer treatment characteristics (chemotherapy, type of radiation treatment, and surgical management), and physical therapy evaluations (circumferential measurements, bioimpedance spectroscopy data, follow-up) can be included in the analysis.

An ALND procedure includes resection of axillary level I and II nodes. Patients undergoing an ALND can undergo identification of divided lymphatics with FITC and subsequently re-route those channels into a preserved axillary vein tributary.

Demographics and potential risk factors for development of lymphedema such as age, body mass index, clinical stage, radiotherapy, and chemotherapy were reviewed. Similarly, patients who underwent the LYMPHA technique were compared to those who only had ALND.

All p-values were computed using the Fisher Exact Test or two-tailed t-test, as appropriate. Computations were done in the R language for statistical computing, version 3.3.2. A power analysis can be performed using SAS with the Fischer's Exact Conditional Test, for example. This utilized a set control percentage of 0.40 based on our institutional data. As previously noted, the incidence of lymphedema after simultaneous lymphovenous bypass was 0.04. Conservatively, in evaluating this procedure the power can be set at 0.8.

In a study conducted by Hahamoff et al (“A Lymphatic Surveillance Program for Breast Cancer Patients Reveals the Promise of Surgical Prevention”, Journal of Surgical Research, 2017, 10.008, the entire contents of which is incorporated here by reference) 177 patients presented for a pre-operative lymphedema evaluation and 87 patients (49%) participated in the program over the period. 45% (67/145) of patients undergoing sentinel lymph node (SLN) biopsy and 64% (18/28) of patients undergoing ALND participated in the program and had an average age of 60 (range 32-83) and BMI 30 (range 17-46). 40% underwent a mastectomy and 21% underwent an ALND. 18% received neoadjuvant chemotherapy and 24% received RLNR. Most patients in this example did not undergo any reconstruction (62%).

The single most significant risk factor for the development of lymphedema was an ALND (p<0.001). Undergoing mastectomy (p=0.02), adjuvant chemotherapy (p-0.03), and RLNR (p=0.05) were also associated with lymphedema development. A trend towards lymphedema development and clinical stage III discase (p=0.10) was also noted.

TABLE 1
Advantages and disadvantages of the two most commonly
used fluorophores in lymphatic surgery (Blue Dye and ICG)
in comparison to FITC.

| Dye | Advantages | Disadvantages |
|---|---|---|
| Blue Dye | Technical | Technical |
| | ✓Visualized through | [symbol] No Depth of Penetration |
| | Binoculars | [symbol] Permanent Staining |
| | (Live Surgery) | Safety |
| | ✓No Specialized | [symbol] Adverse Reactions |
| | Equipment Necessary | Skin Necrosis (Methylene |
| | | Blue) |
| | | Anaphylaxis (Isosulfan Blue) |
| | | [symbol] Cross Reactivity |
| | | Sulfa Drugs (Isosulfan Blue) |
| | | SSRI (Methylene Blue) |
| ICG | Technical | Technical |
| | ✓Depth of penetration = | [symbol] Unable to visualize through |
| | 20 mm | binoculars (No Live Surgery) |
| | Safety | [symbol] Permanent staining |
| | ✓No adverse reactions | Requires Specialized |
| | (dermal) | Equipment |
| | ✓No cross-reactivities | |
| FITC | Technical | Technical |
| | ✓Visualized Through | [symbol] Requires Specialized |
| | Binoculars | Equipment |
| | (Live Surgery) | |
| | ✓Depth of penetration = | |
| | 5 mm | |
| | No permanent staining | |
| | Safety | |
| | ✓No adverse reactions | |
| | (dermal) | |
| | ✓No cross reactivities | |

All patients who developed lymphedema were initially diagnosed either during treatment or within six months of the completion of their cancer therapy. Therefore, all patients were initially diagnosed with transient lymphedema. The average time to diagnosis after the surgical procedure was 4.7 months. One patient in the SLN biopsy group developed transient and then persistent lymphedema (1/67 or 1.5%). Of five patients who developed transient lymphedema after undergoing an ALND without the LYMPHA procedure, one patient's symptoms and objective measures completely resolved and four patients' symptoms persisted and they developed lymphedema ( 4/10 or 40%). Of these four patients, three were diagnosed with lymphedema based on changes in symptoms with associated changes in circumferential measurements and bioimpedance spectroscopy. The fourth patient was diagnosed based on symptoms and changes in circumferential measurements alone. Of the 17 patients who underwent the LYMPHA procedure during the period, only eight participated in our surveillance program. One patient in the ALND+LYMPHA group developed transient lymphedema which was persistent but still within six months of the completion of adjuvant radiation therapy (⅛ or 12.5%). This patient's diagnosis was based on changes in symptoms and bioimpedance without change in circumferential measurements. The only significant difference between the two groups undergoing ALND with or without LYMPHA was the follow-up period of 15 months versus 20months (p<0.03), respectively.

In a comparison of patients who underwent ALND with or without LYMPHA versus those lost to follow-up in order to identify any potential confounding factors or bias, the only difference between groups noted is that participants who underwent LYMPHA were 10 years older than those patients lost to follow-up (59 vs 49, p=0.04).

With no cure to date for BCRL, recognition and prophylactic treatment for high-risk patients is an important consideration. The rate of lymphedema after ALND can be reduced from 40% to 12.5% after introduction of the LYMPHA approach in this example. Similarly, it is preferable to identify lymphedema in patients undergoing ALND within five months of their procedure. ALND, mastectomy, adjuvant chemotherapy, and RLNR were associated with the development of lymphedema.

A notable finding of the Hahamoff et al, study was the reduction in rate of lymphedema development from 40% to 12.5% in patients undergoing an ALND after the introduction of the LYMPHA technique.

Note that the patients who develop lymphedema presented initially with signs and symptoms either during treatment or within six months of the end of their cancer therapy. Of these patients, one patient's condition completely resolved. No patient, to date, has presented with lymphedema more than six months after the completion of cancer therapy. This finding underscores the value of surveillance in being able to detect early lymphedema which is especially important for high-risk patients as prompt detection and treatment can potentially slow the progression of disease.

ALND and RLNR are important risk factors for the development of lymphedema. There can be increased rate of lymphedema in patients undergoing mastectomy, and this can be explained by the indications for ALND. Specifically, patients with limited nodal involvement undergoing lumpectomy do not require an ALND while those undergoing mastectomy will undergo an ALND for the same extent of nodal involvement. Therefore, patients undergoing mastectomy receive more aggressive axillary management than those undergoing lumpectomy. There can be increased rates of lymphedema for patients who underwent adjuvant chemotherapy, which again, may be biased as those undergoing chemotherapy are more likely to have presented with more advanced disease initially. However, studies have linked specific chemotherapy regimens to the development of lymphedema. Lastly, as patients presenting for ALND have more advanced disease, it is not surprising that increased rates of lymphedema development were noted in those with clinical stage 3 disease.

While surgical prevention can aid in improving the quality of life in breast cancer survivors, development of this program did have challenges. When SLNs were sent for permanent section and the patient returned to the operating room for an ALND at a later date, scheduling combination procedures between a breast and plastic surgeon were effective. However, when SLNs were sent for frozen section, the scheduling can be more erratic as a larger percentage of patients will never progress to ALND especially in light of recent trials challenging the need for ALND.

The present devices and methods for the treatment of lymphedema can change how metastatic disease to the axilla is treated. Given the significant morbidity of ALND, namely lymphedema, there is a distinct push away from ALND in early stage breast cancer in place of RLNR. However, with improved LYMPHA procedures and the promise of lower rates of lymphedema, the role of ALND in providing an improved method of loco-regional control can be enhanced.

A significant finding was that a decrease in lymphedema rates after the advent of LYMPHA are notable as the average time to diagnosis of lymphedema was 4.7 months following the surgical intervention. In this example, the total follow-up time in the ALND versus ALND+LYMPHA groups was 20 months and 15 months, respectively.

Offering LYMPHA with ALND together decreased the rate of lymphedema from 40% to 12.5%. Similarly, surveillance after surgery can provide early diagnosis and intervention by physical therapy. The significant risk factors for lymphedema development included ALND, RLNR, adjuvant chemotherapy, and mastectomy.

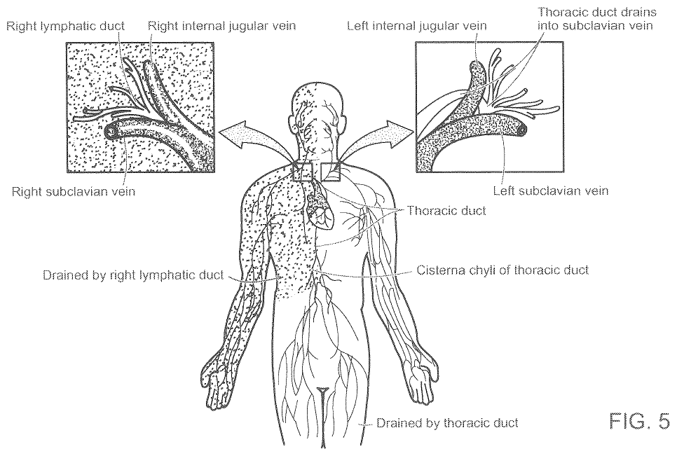

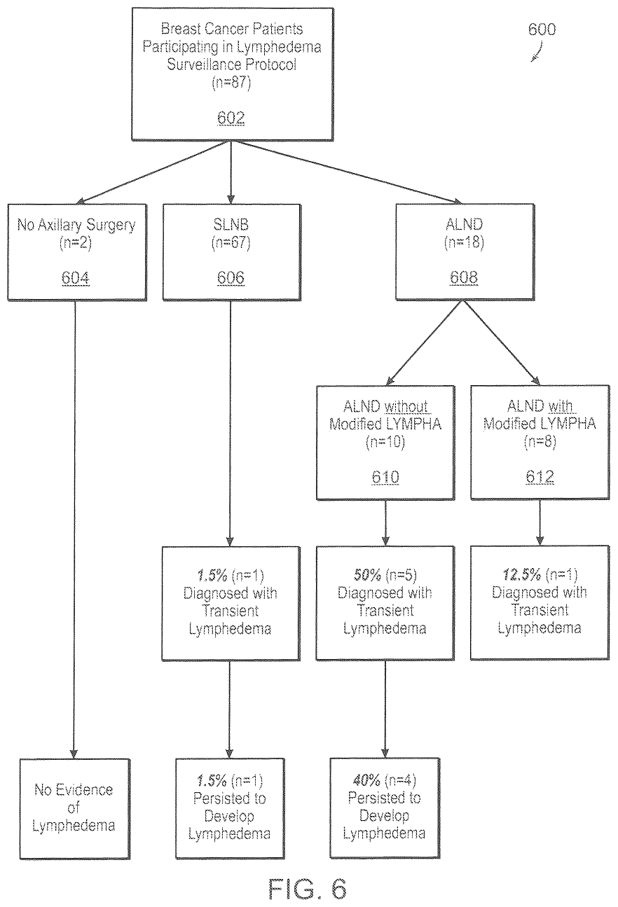

Note that breast surgeons often prefer to use a dual tracer method including both blue dye and technetium sulfur colloid for sentinel lymph node (SLN) identification. This is especially important in cases where neoadjuvant chemotherapy has previously been administered. Therefore, a different dye was sought for arm lymphatic mapping to differentiate staining from arm versus breast lymphatics. Thus, a combination of visualization procedures can be used. Shown in FIG. 5 are regions of the body containing portions of the lymphatic system. Each of these regions can be imaged to map the flow of lymph as needed for a particular condition.

The most common method of lymphatic vessel mapping currently in use is indocyanine green (ICG). However, the challenge with ICG is that the dye is near-infrared and therefore excited in the non-visible spectrum. This limits the usefulness of ICG for visualization and simultaneous dissection as the dye is displayed as a white signal on a black background and cannot be concurrently visualized through the binoculars of a microscope.

Illustrated in FIG. 6 is a flowchart 600 exhibiting the steps associated with treating lymphedema in cancer patients. The process is initiated at 602 where patients can be routed through one of three distinct protocols 604, 606, 608. In the first protocol 604, no axillary surgery is performed and follow up indicates that there is no observed lymphedema. The second protocol 606 employs sentinel lymph node biopsy where a certain population develops lymphedema requiring treatment. A third protocol 608 involved performing an ALND procedure either with 612, or without 610, a LYMPHA procedure as described herein.

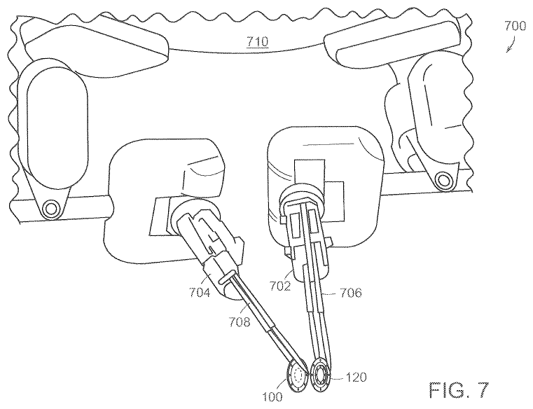

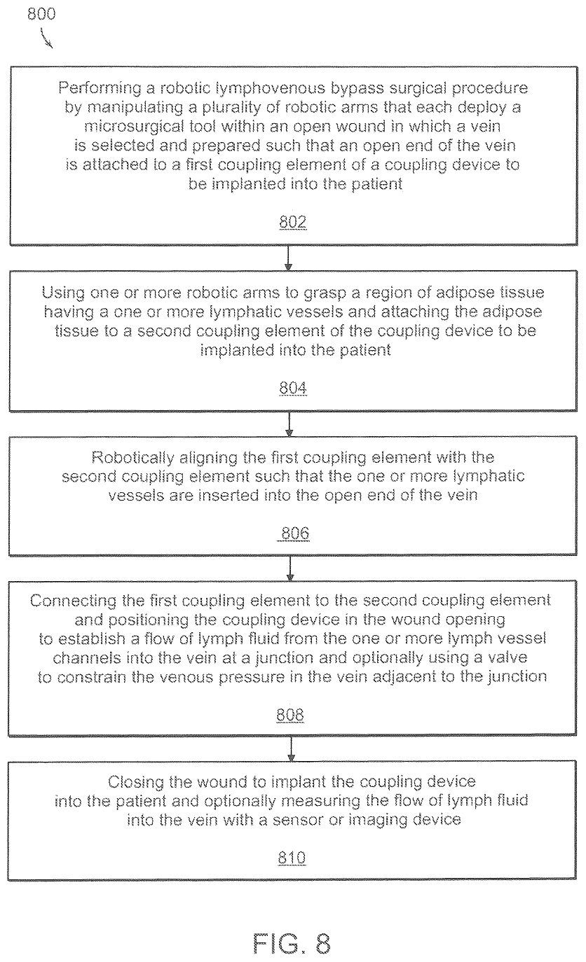

Illustrated in connection with FIGS. 7 and 8 are systems and methods for performing a robotic lymphovenous bypass surgical procedure for implanting a coupling device as described herein. Robotic systems such as the Da Vinci system available from Intuitive Surgical Inc., Sunnyvale CA, have been used to perform the LVA microsurgical procedure as illustrated in connection with FIG. 3. See van Mulken et al, “First-in-human robotic supermicrosurgery using a dedicated microsurgical robot for treating breast cancer-related lymphedema: a randomized pilot trial”, Nature Communications, 11:757, Feb. 20, 2020, the entire contents of which is incorporated herein by reference. Further details concerning robotic surgery are described in U.S. Pat. No. 9,138,297, the entire contents of which is incorporated herein by reference. This system 700 can employ robotic arms 702, 704 attached to grasping elements 706, 708 such as forceps-like manipulators. A surgeon can use the system 700 to grasp and control microsurgical tools within the surgical field. The computerized system 710 in the system 700 is programmed with software to perform scaling motion and tremor filtration, for example. As described in the process flow diagram of FIG. 8, the process 800 uses a plurality of two or more control arms 702, 704 that are actuated to perform the procedure wherein a vein is selected 802 having a diameter suitable for coupling to a first (or second) coupling element as described herein. The robotic arms can further grasp a region of adipose tissue having one or more lymphatic vessels wherein the adipose tissue is attached 804 to the second (or first) coupling element as described herein. The robotic arms can grasp the first and second coupling elements 100, 120, as seen in FIG. 7, to align the two elements such that the lymphatic vessels are inserted into the vein 806, typically under surgical microscope visualization. The robotic grasping tools 706, 708 can hold the two coupling elements by the outer peripheral surfaces which may be made with notches or slots to enable a stable and secure grip. The two coupling elements are connected 808 to each other and the device is positioned within the wound opening for closing 810 of the wound.

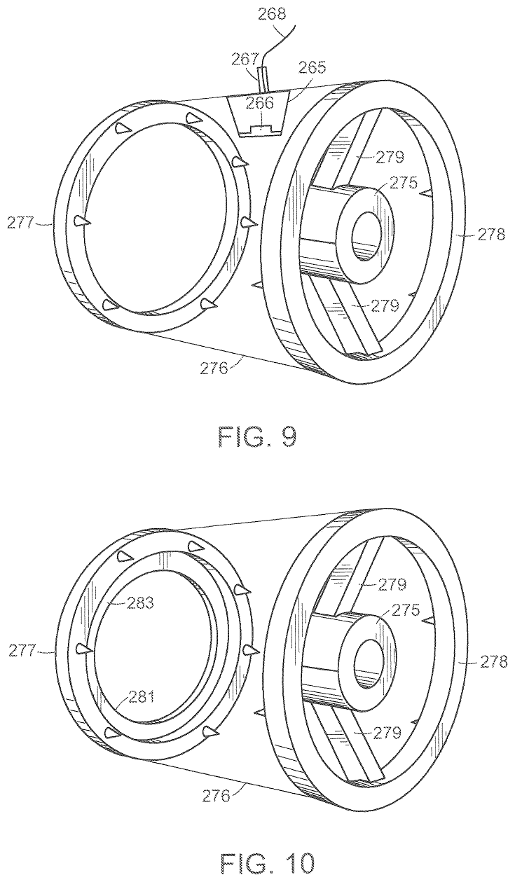

As shown in FIG. 9, one or more sensors 265 or imaging devices can be used to measure the flow of lymph fluid into the vein at the junction within the device. The sensor 265 can be an optical sensor, for example, wherein a light source such as a light emitting diode (LED) or laser diode can be positioned relative to a photodetector array within a sensor module 266 that contacts the outer surface of the vein. As described herein, a fluorescent dye can be delivered into the lymphatic vessels prior to, during or after the procedure such that the optical sensor can measure the flow rate by detecting movement of the dye. Alternatively, the sensor 265 can comprise an ultrasound transducer 266 that can transmit an acoustic signal into the vein to detect reflectors that are introduced into the lymphatic vessels with the fluorescent dye. A cable or wire 268 can extend through a transcutaneous port 267 that extends to the tissue surface after wound closure. The cable is connected to a computer controlled data processing and display device for viewing of the measured data on the display and for storage of the data in a memory. This data can be transported to the electronic medical record for each patient. The sensor can be sized and configured to be inserted through the port in certain embodiments so as to enable easy insertion and removal after wound closure. As described previously, the sensor and/or fluoroscopic imaging can optionally be used during and/or after the procedure to verify proper positioning of the lymphatic vessels and lymph flow. The device can optionally also be coated with one or more therapeutic agents that inhibit clot formation within the vein in proximity to the junction. Shown in FIG. 10 is a further embodiment in which a flexible valve ring 281 can be attached to the coupling element 277 with a membrane 283. The inner surface of valve element 281 is in contact with the outer surface of the vein that is attached to the pins shown on inner surface of element 277. The valve element can be sized to constrict the vein so as to limit venous pressure on the junction within the device so as to decrease backpressure from the vein fluid on the junction region. This reduced pressure at the junction can aid in establishing flow of lymph fluid which tends to increase over time. The valve element can be shaped, sized and configured to accommodate the slow increase in lymph fluid pressure at the junction and can reduce the amount of compression over time. The valve element can comprise a biodegradable material that eases the constraint on the vein overtime due to the rate of degradation of the material. The valve can also be active, such as by a pressurized bladder that can release a pressurized fluid such as saline over time. Alternatively, pliable flaps can also impart sufficient pressure on the vein with an elastic material that expands at a selected rate.

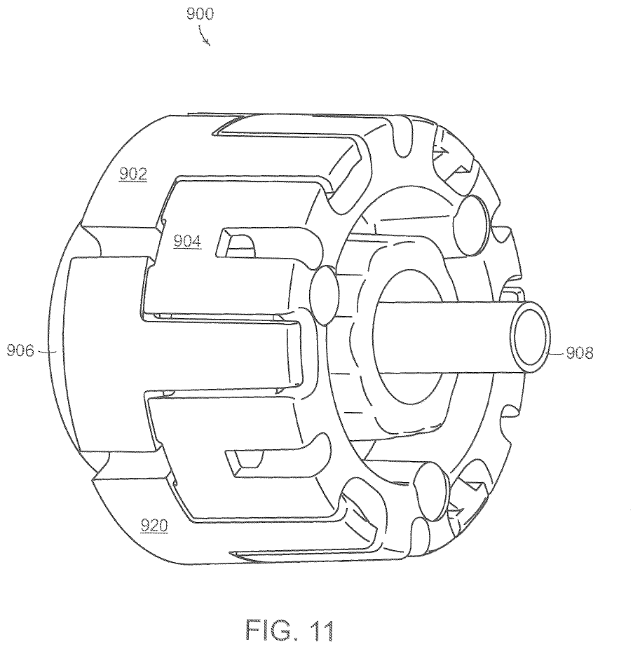

Shown in FIG. 11 is a further embodiment in which a coupling device 900 can have a first coupling element 902 with a first ring outer surface 920 and a second coupling element 904. The first coupling element 902 can be attached to adipose tissue 906 including lymphatic vessels 905 while the second coupling element 904 can be attached to a vein 908. When the first coupling element 902 and second coupling element 906 are brought together to form the coupling device 900, lymph fluid from the lymphatic vessels 905 is drained in to the vein 908.

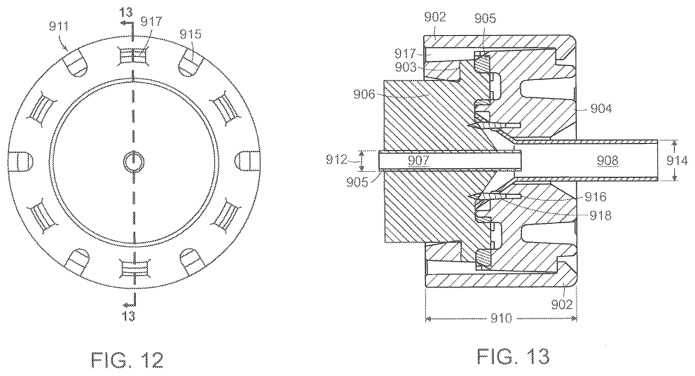

FIG. 12 illustrates a bottom view of coupling device 900. The first coupling element can be in the form of a ring element having a first ring outer surface 920 and connecting element prongs 922. The second coupling element 904 can include a cone shaped element that can first engage the vein that is coupled to the pins and also receive adipose or fat tissue 906 within the cone volume, such that one or more lymphatic vessels 905 can be inserted into the vein. The adipose tissue 906 and embedded lymphatic vessels 905 can extend through ring opening 901 of the coupling device 900. The lymphatic vessels 905 can be inserted into the vein 908 which can extend through an opening in the second coupling element 904. In some embodiments, the first coupling element 902 can be connected to the second coupling element 904 via connecting elements 922. The connecting elements 922 of the first coupling element 902 can engage with receiving elements on the second coupling element 904 thereby connecting the first coupling element 902 to the second coupling element 904. In some embodiments, the second coupling element 904 can have a tissue grasping elements 918 that can be positioned to grasp the end of a vein 908. In some embodiments, the tissue grasping elements 918 can be pins. The pins 918 can be formed integral with the second coupling element or can be inserted into openings 916 in the second coupling element. The tissue grasping elements 918 of the second coupling element 904 secures vein 908 in place during an operation. The first coupling element 902 and second coupling element 904 can comprise a rigid or semi-rigid compliant, or elastic biocompatible material with generally smooth surface features except for the pins, prongs or tissue anchors that are configured to penetrate and grasp tissue. In some embodiments, the first coupling element 902 and second coupling element 904 can have surfaces that can allow the two components to snap together more easily to form coupling device 900.

As shown in FIG. 12, the first coupling element 902 can have connecting element prongs 922 that are configured to connect the first coupling element 902 to the second coupling element 904 to form coupling device 900. In some embodiments, the first coupling element 902 can have ring wall channels 911 that can provide surface regions that enable a user to grasp the element using a tool and maneuver the coupling element or device 900. When joined together, the surface 915 of the second ring outer ring wall channels 911 of the second coupling element 904 abuts the end of each ring wall element in the first coupling element 902.

FIG. 13 illustrates a cross-sectional view through the coupling device 900 taken along the line illustrated in FIG. 12. As shown in FIG. 13, the lymphatic vessel wall 905 can extend into an interior 908 of the vein thus allowing lymph fluid to drain from the lymphatic vessel into the vein. In accordance with various embodiments, the second coupling element 904 grasps the vein. The vein can have a diameter 914 in a range of 1-3 mm, for example. In some embodiments, first coupling element 902 can have a tissue contact surface 903 that can provide support for contact between first coupling element 902 and adipose tissue 906. In some embodiments, a diameter of the opening 936 in the first inner ring can be in a range between 5 mm and 12 mm.

In other embodiments, the opening 936 can have a diameter less than 7.2 mm or greater than 10 mm. In some embodiments, the first coupling element 902 can have a length 910 in a range of 5-10 mm and preferably about 7 mm. The diameter of the opening 936 can be large enough to comfortably pass micro-forceps (typical tip width of 0.5 mm for each arm of the micro-forceps) through the opening, grasp tissue, and pull tissue through the opening 936. In conventional devices for vein-to-vein anastamosis, a small diameter opening is provided on both elements to enable a vein to pass through. Such devices have openings that may be too small to pass micro-forceps and too small to pull bundled adipose tissue and lymphatic channels therethrough. Systems and methods described herein can employ larger diameter openings 936 to facilitate tissue manipulation and to ensure that one or more lymphatic vessels are positioned relative to the vein so as to drain lymph fluid into the vein.

In some embodiments, the first coupling element 902 can attach to adipose tissue 906 that includes one, two, or more lymphatic vessels. A single lymphatic vessel is illustrated in FIGS. 11-15. The lymphatic vessel wall 905 can have a channel 907 within the lymphatic vessel wall 905. In some embodiments, the channel 907 within the lymphatic vessel wall 905 can have a diameter 912 of about 15-4000 microns (0.015-4 mm) or, more preferably, in a range from 0.1-0.8 mm. In applications where lymphatic trunk vessels are coupled to a vein using devices of the present disclosure, the diameter 912 of the lymphatic vessel can be in a range from 2-4 mm. The first inner ring 901 can contact the adipose tissue 906 between first coupling element 902 and second coupling element 904. In some embodiments, there can be open channels 917 through first inner ring 901. In some embodiments, the second coupling element 904 can have pin securing regions 918 for pins 916. The pin securing regions 918 can include holes that each connect to a portion of the pin 916 to be secured. The pin securing region 918 can include threaded holes in some embodiments. The pins 916 secure the vein to second coupling element 904. The pins 916 are described in greater detail below with respect to FIG. 21.

In preferred embodiments, the channel 907 within the lymphatic vessel can extend a distance within the vein 908 when the coupling device 900 is assembled. In other words, the lymphatic vessel can be intussuscepted into the vein. In some embodiments, the channel 907 can extend a distance of 0.5 mm, 1 mm, 1.5 mm, or more. By extending the channel 907 into the vein by a distance, lymph fluid exiting the channel is coupled directly into the vein 908 and can be drained. In other embodiments, the channel 907 does not extend into the vein when the coupling device 900 is assembled. Rather, the ends of the lymphatic vessel and the vein can be aligned and sealed within the coupling device 900. After assembly, lymph fluid may continue to leak from the lymphatic vessel and contact adipose tissue 906, which can fill the interior space within the coupling device 900. After contact with lymph fluid, the adipose tissue 906 may be changed to a natural lining material such as occurs in seroma cavities. This lining material is relatively impervious to penetration of further lymph fluid. The generation of this lining material on surfaces where lymph fluid is contacted creates a seal within the coupling device 900 that prevents lymph fluid from exiting by any means other than through the vein 908.

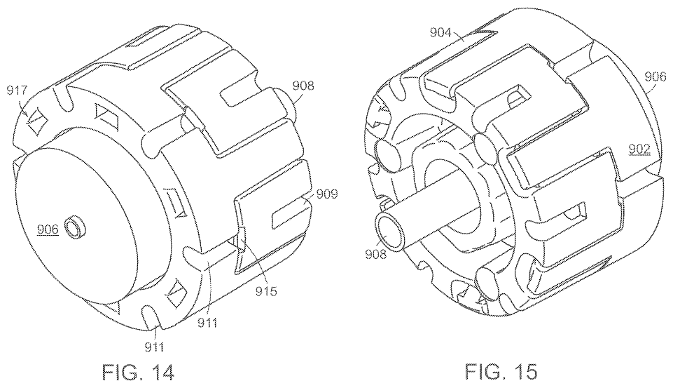

FIGS. 14 and 15 show front and rear perspective views of coupling device 900, respectively. As noted above, the first coupling element 902 can have ring wall channels 911 that can provide structural support and maneuverability to the coupling device 900. In some embodiments, ring wall channels 911 can assist in the securing of first coupling element 902 to second coupling element 904. In some embodiments, second coupling element 904 can include interior cylindrical wall channels 909. The interior cylindrical wall channels 909 can provide flex while maintaining a lighter overall weight for coupling device 900. In some embodiments, the wall channels 909 can be engaged by a tool to enable holding and manipulation of the second coupling element 904 during implantation. In some embodiments, coupling device 900 can have surface 915 of a second ring outer ring wall channel 911. In some embodiments, coupling device 900 can have open channels 917 through ring hole 901. The open channels 917 can enable tissue ingrowth upon implantation of the coupling device 900 that provides additional stabilization of the device over time.

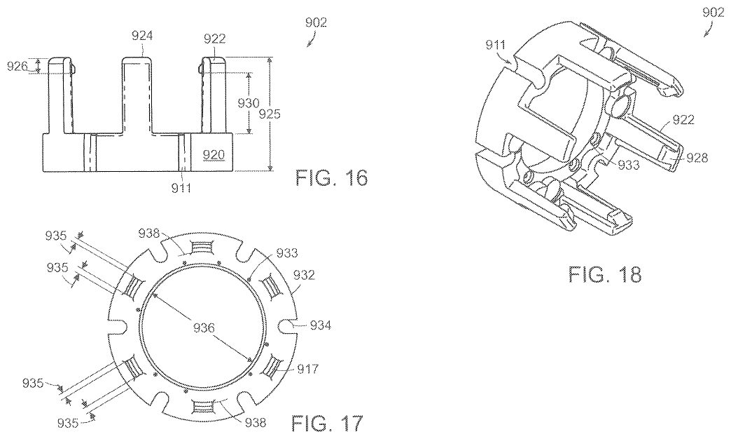

As shown in FIG. 16, first coupling element 902 can have first ring outer surface 920. In some embodiments, the first ring outer surface 920 extends to connecting element prongs 922. In some embodiments, the connecting element prongs 922 can have a top surface 924. In some embodiments, the top surface 924 of the connecting element prongs 922 can be rigid and textured. In some embodiments, the connecting elements 922 can allow for a snap like connection between the first coupling element 902 and the second coupling element 904 to form coupling device 900. In some embodiments, the first coupling element 902 can have a height 925 of about 7 mm. In some embodiments, the distance 930 between the bottom of the hooks or latch elements 928 and the ring can be about 4 mm. In some embodiments, the connecting elements 922 can have a width 926 of about 1 mm.

As shown in FIG. 17, the opening 936 in the first coupling element 902 can have a diameter in a range from 5 mm to 15 mm. In some embodiments, the first coupling element 902 can have ring wall channels 911 with diameter 934 of about 1 mm. In some embodiments, the first coupling element 902 can have a diameter 938 between opposing indentations in the ring base.

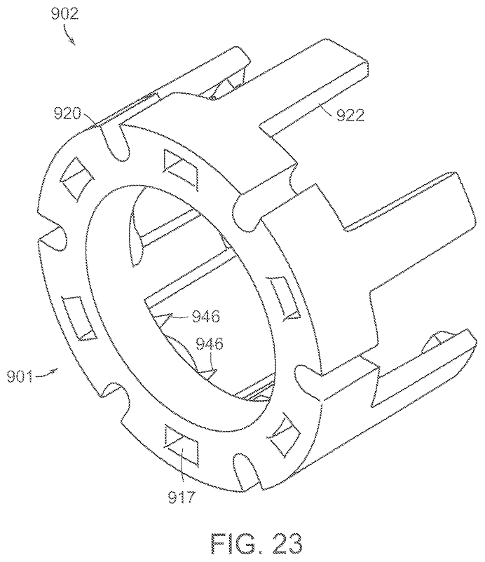

As shown in FIG. 18, the connecting elements 922 can include hooks or latch elements 928. In some embodiments, the first coupling element 902 can include pin securing regions 933. The pin securing regions 933 can accept and secure pins 946 (shown in FIG. 23, for example). Pins 946 may be similar to the pins 918 associated with the first coupling element 902. The pins 946 at pin securing regions 933 can connect to or grasp adipose tissue 906 containing lymphatic vessels. For example, adipose tissue 906 can be brought through the opening 936 of first coupling element 902 and stretched over one or more of the pins 946.

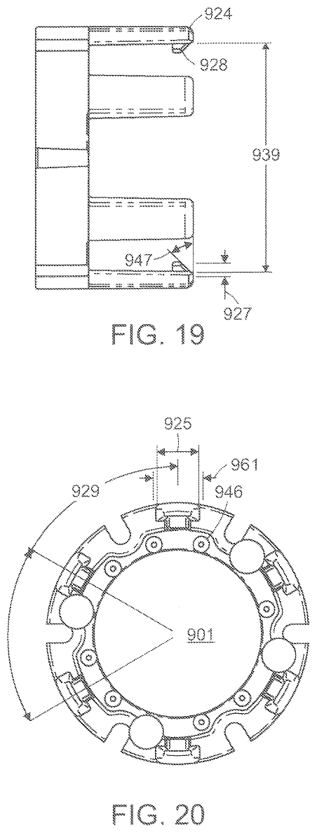

As shown in FIG. 19, the first coupling element 902 can have latch elements 928 on the connecting element prongs 922 having the top surface 924. In some embodiments, the distance 939 is between inner surfaces of opposite connecting elements 922. In some embodiments, the latch elements 928 can extend from the inner surface of the connecting elements 922 by a distance 927. In some embodiments, the angle 947 of an upper surface of the hook or latch elements 928 can be about 40-65 degrees, for example.

As shown in FIG. 20, first coupling element 902 can have an angular distance 929 between neighboring connecting element prongs 922 of about 60°. In some embodiments, the width 925 of each latch 928 can be about 1-2 mm, for example. In some embodiments, a width 961 of each prong is larger than the corresponding latch.

As shown in FIG. 21, in some embodiments pins 918 can have a fastener 913 at the base of the pin 918. In some embodiments, the fastener 913 can be screw threads that mate with pin securing regions 916. In some embodiments, an end of each pin 918 can narrow to a tip 921. In some embodiments, the tip 921 can be pointed. In some embodiments, the tip 921 of pins 916 can hold onto a fatty tissue 906 or a vein wall.

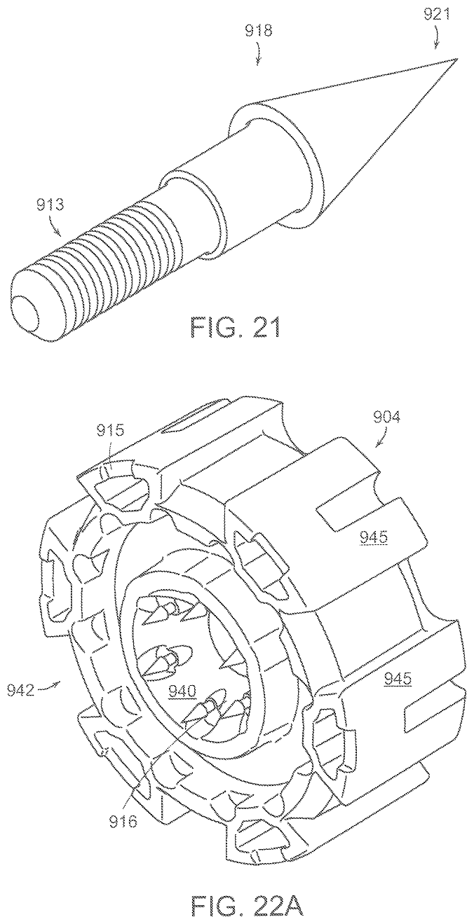

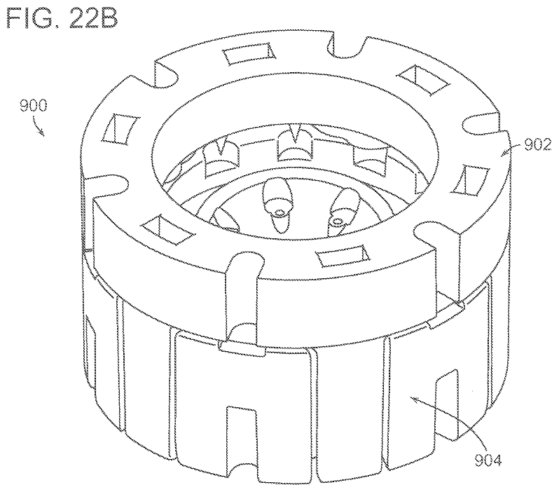

As shown in FIG. 22A, second coupling element 904 can have ring channels 942. The central portion of the second coupling element 904 can include a cone shaped surface 940. The cone shaped surface 940 can increase in diameter from the narrowest diameter at a bottom of the second coupling element 904 to the largest diameter at a top surface of a raised ring. In some embodiments, the cone shaped surface 940 can receive fatty tissue 906. In some embodiments, the pins 916 can be positioned in pin securing region 918. The pin securing regions 918 can be located on the cone-shaped surface 940 such that pins 916 will extend from the cone-shaped surface 940. In some embodiments, second coupling element 904 can have a second ring outer surface 945. In some embodiments, cone-shaped surface 940 can be elevated above the surface of second coupling element 904 by one or more millimeters. This can provide for the insertion of lymphatic channels 907 to be a depth of at least 1 mm into the vein 908, for example. Thus, the relative dimensions of the coupling elements can define a depth of insertion.

A perspective view of an exemplary coupling device 900 is shown in FIG. 22B. When the first coupling element and second coupling element are connected, an interior volume is created within the coupling device. The interior volume can be filled with adipose tissue that is under some tension, having been pulled through the ring aperture. The adipose tissue can work to seal the ring aperture to prevent lymph fluid from escaping the interior volume.

As shown in FIG. 23, first coupling element 902 can have coupling element prongs 922 that can connect a second coupling element 904 to form coupling device 900. In some embodiments, first coupling element 902 can have a first ring outer surface 920 of first inner ring 901. First coupling element 902 can include pins 946 to connect to and grasp adipose tissue 906.

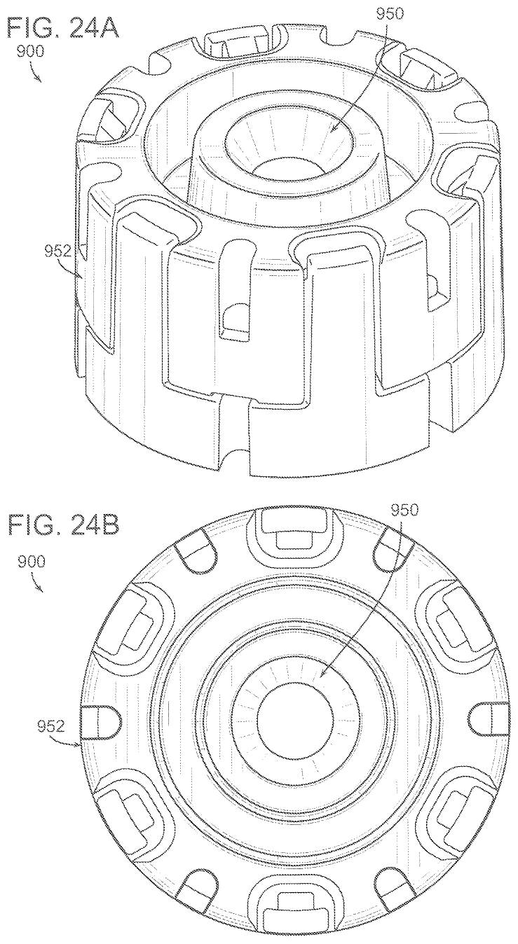

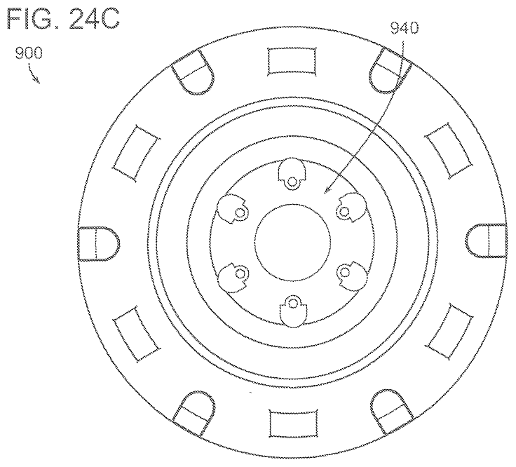

As shown in FIGS. 24A-24C, the coupling device 900 of some embodiments can be defined by a cylindrical wall 950 that surrounds the components. As noted above, in some embodiments the coupling device 900 can be formed by connecting a first coupling element and a second coupling element. The first coupling element and the second coupling element can be separate pieces or can be joined using a connecting sheath, hinges, or other connecting devices that allow the elements to move with respect to one another. Alternatively, the coupling device 900 can be formed as a single integral object. In some embodiments, the coupling device 900 can have a first cone surface 952 and a second cone surface 940. The first cone surface 952 can assist with placement of the vein through into the device and avoid abrasion of the tissue. The second cone surface 940 can provide support as the vein diameter widens from the usual diameter to an expanded diameter at a top edge of the second cone surface 940. Tissue grasping elements can extend from the second cone surface to secure tissue such as a vein.

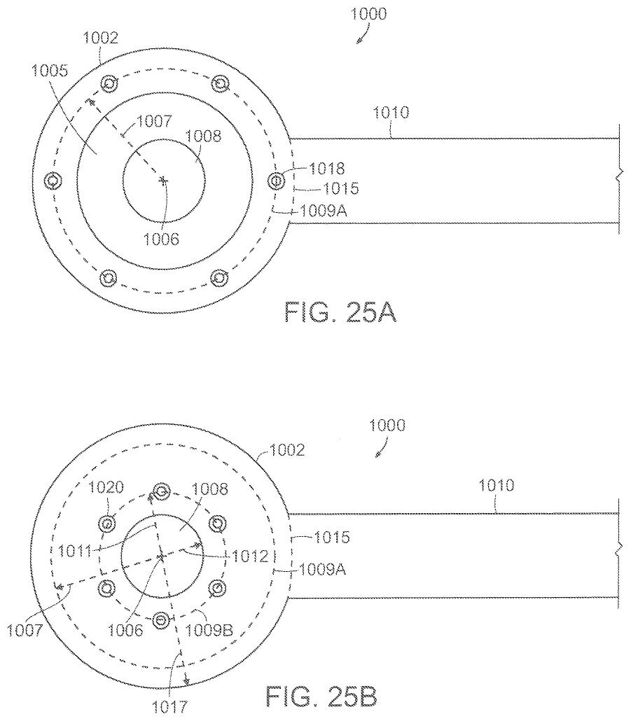

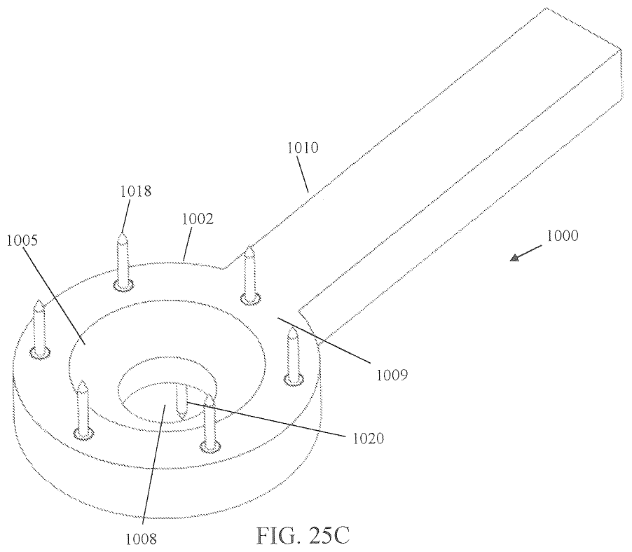

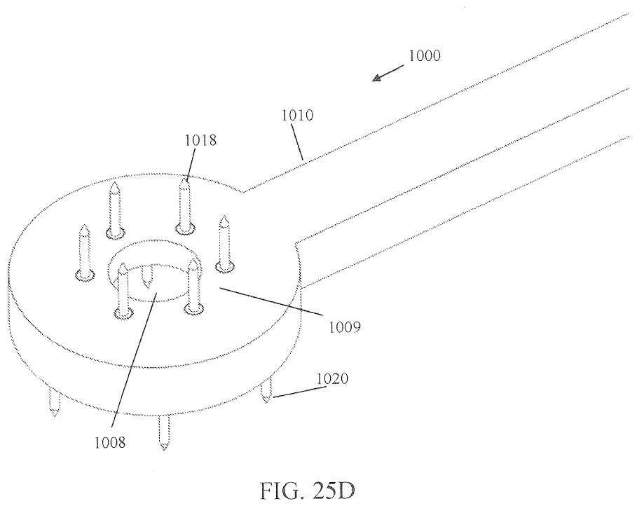

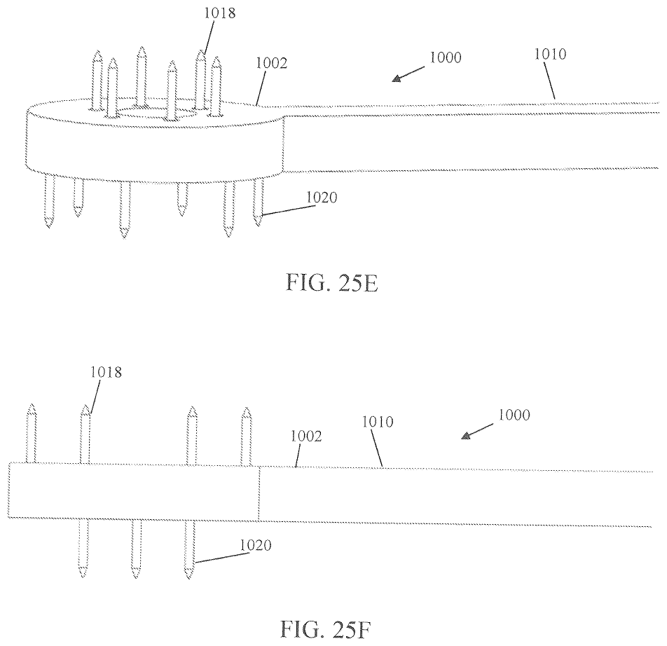

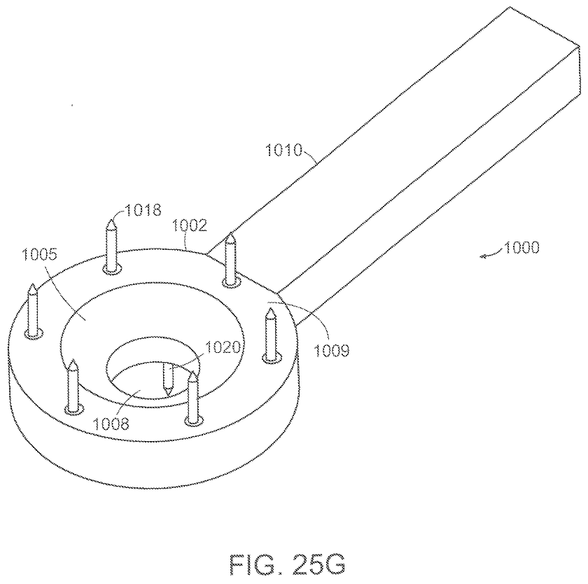

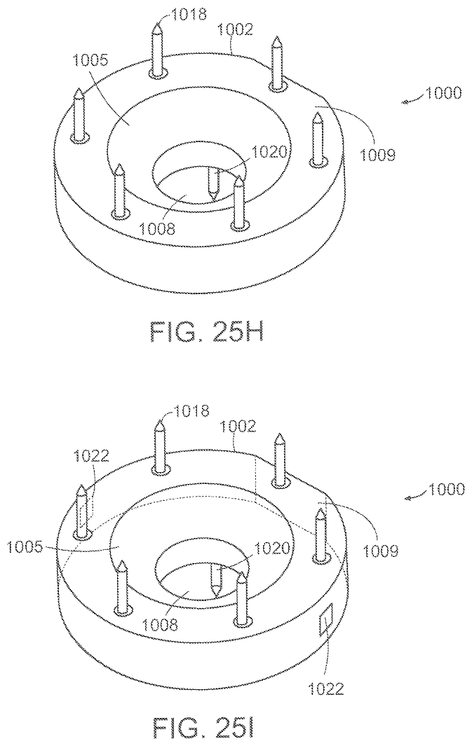

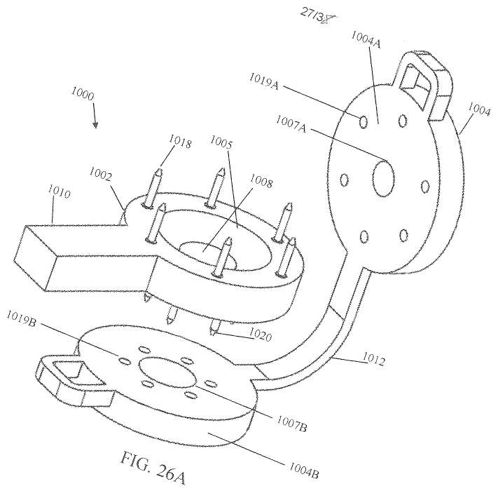

Shown in FIGS. 25A-251 is a further embodiment in which a coupling device 1000 can have a first coupling element 1002. In this embodiment, the first tissue grasping element(s) 1018 for grasping the lymphatic channels and the second tissue grasping element(s) 1020 for grasping a vein or artery are positioned on a single element referred to herein as the first coupling element. The first coupling element 1002 may have an aperture 1006 extending through a plane having a first side and a second opposing side of the first coupling element 1002. The plane can be parallel to a first ring surface on which the first tissue grasping elements are affixed and extend on one side of the plane from the first ring surface. The first coupling element 1002 can include first tissue grasping elements 1018 extending in a first direction from a first side of the plane and configured to attach to tissue. For example, the first tissue grasping elements 1018 may attach to lymphatic tissue, such as lymphatic channels, and/or the fatty tissue surrounding the lymph channels. The first coupling element 1002 may include second tissue grasping elements 1020 extending in a second direction from the second opposing side or second ring surface. The second tissue grasping elements 1020 can be configured to attach to a vein or artery. In some embodiments, the grasping elements 1018 and 1020 may be pins or prongs as described previously herein that are structures configured to facilitate attachment to tissue.

In some embodiments, the first coupling element 1002 has a cone-shaped surface 1005. In further embodiments, the cone-shaped surface 1005 extends around the aperture 1006 of the first coupling element 1002 along a central axis 1006 that is orthogonal to the plane. In one embodiment, the plane extends through the center of the ring midway between the first ring surface and the second ring surface. A smaller cone shaped surface can also be used on the opposite side of the first coupling element as in other embodiments described herein.

The aperture 1008 of the ring can have a radius 1012. The first coupling elements 1018 can extend circumferentially around the aperture 1008 and axis 1006 at a radius 1007. The outer peripheral wall of the ring can have a radius 1017 such that the ring has a diameter within the range of the embodiments previously described herein. The second tissue grasping elements 1020 can also extend circumferentially around the aperture at a radius 1011. Note that radius 1011 of the second tissue grasping elements predominantly smaller than the radius 1007 of the first tissue grasping elements. The tissue grasping elements extend orthogonally from the respective ring surfaces, however, other embodiments can include tissue grasping elements at different oblique angles relative to one or both ring surfaces to better stabilize the tissue for healing. The tissue grasping elements can be of different lengths and diameters as described previously herein.

In some embodiments, the first coupling element 1002 comprises a circle 1009A having the first radius defining positions of the first tissue grasping elements 1018 equally spaced around a first circumference defined by the first radius. In further embodiments, the first coupling element 1002 comprises a smaller circle 1009B having the second radius defining positions of the second tissue grasping elements 1020 equally spaced around a second circumference of the second radius. In some embodiments, the circular geometry 1009A and 1009B may not be physical components of the first coupling element 1002 but may instead refer to the positioning of the tissue grasping elements 1018 and 1020 that may be formed by molding of one or more polymer materials to form a unitary component. In some embodiments, the circle 1009B is sized, shaped, and positioned outside the widest portion of the cone-shaped surface 1005.

Components of the coupling device 1000 may be composed of biocompatible material, for example biocompatible polymer material or silicone, as discussed herein above.

First coupling element 1002 may be connected to a handle 1010. In some embodiments, such as the embodiment depicted in FIG. 25G, handle 1010 may be breakably attached to the first coupling element 1002. For example, a surgeon may break off handle 1010 after attaching a vein or artery to the first coupling element 1002, after attaching the lymph channels to the first coupling element 1002, or after securing one or more cap elements to the first coupling element 1002. FIG. 25H depicts a first coupling element 1002 after removal of the handle 1010.

In some embodiments, such as the embodiment depicted in FIG. 25I, the first coupling element 1002 does not include a handle. Instead, the first coupling element 1002 may include one or more recesses 1022 configured to be grasped by a surgical tool, for example, a forceps. The recesses 1022 may be situated on or within the outer surface of the first coupling element 1002. In some embodiments, the first coupling element 1002 includes two recesses 1022. In further embodiments, the two recesses 1022 are situated 180 degrees apart from one another.

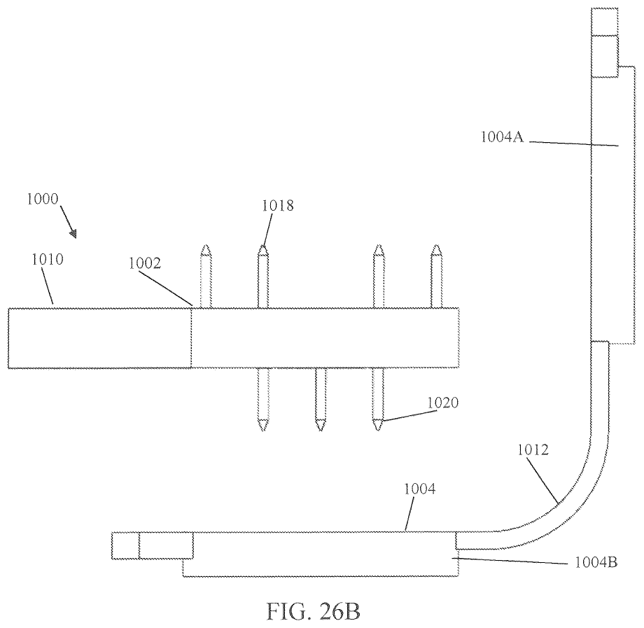

FIGS. 26A-26B illustrate a perspective view of the first coupling element 1002 and a second coupling element 1004 in the form of a connected cap 1012. The second coupling element 1004 may be configured to connect to the first coupling element 1002. Either end of the second coupling element 1004 may include at least one opening 1007A or 1007B configured to receive the vein or artery.

The first coupling element 1002 and the second coupling element 1004 are configured to position one or more lymphatic channels into an open end of a vein or artery of a patient to deliver lymph fluid from the lymphatic channels into the vein or artery. The benefits associated with delivering lymph fluid from the lymphatic channels into the vein or artery are described herein above.

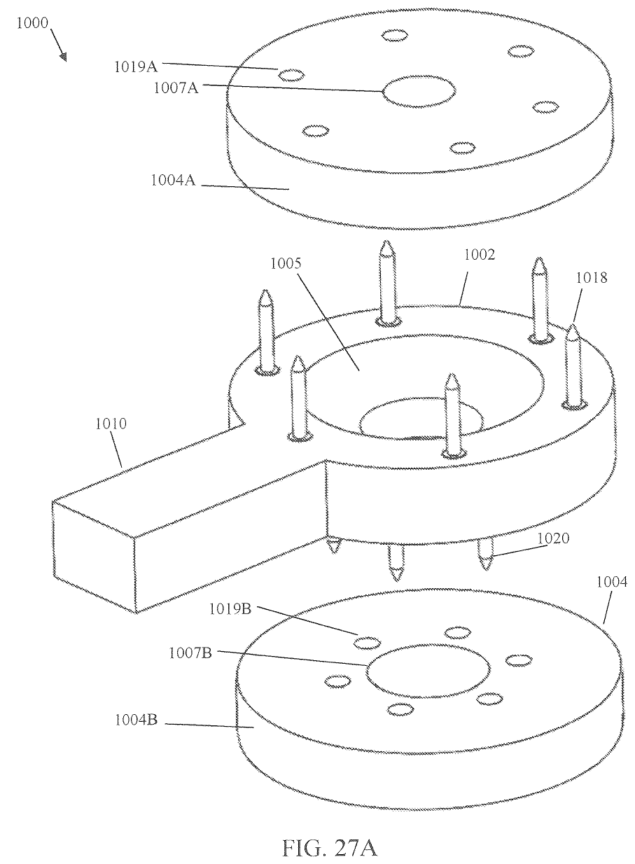

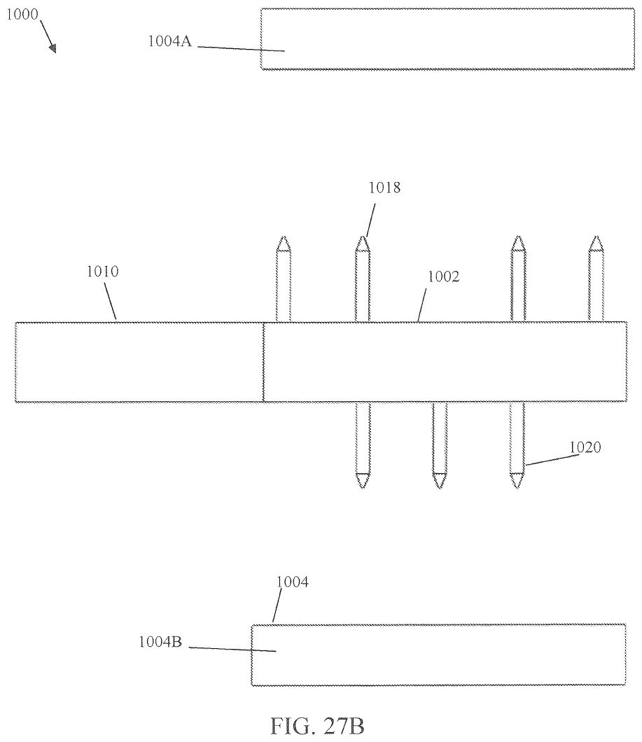

FIGS. 27A-27B illustrate a perspective view of the second coupling element 1004 in the form of two disconnected cap elements. In some embodiments, the second coupling element 1004 may include a first cap element 1004A having a first opening 1007A and a second cap element 1004B having a second opening 1007B. In some embodiments, first opening 1017A has a smaller diameter than second opening 1017B. In some embodiments, first cap element 1004A and second cap element 1004B are of similar diameter.

In some embodiments, the first cap element 1004A encloses the first tissue grasping elements 1018 and the second cap element 1004B encloses the second tissue grasping elements 1020. Accordingly, each cap element may include a plurality of bores 1019A and 1019B configured to receive the first tissue grasping elements 1018 and second tissue grasping elements 1020, respectively. The cap elements can alternatively snap into place with elements that flex to engage ridge elements or edges on the coupling element.

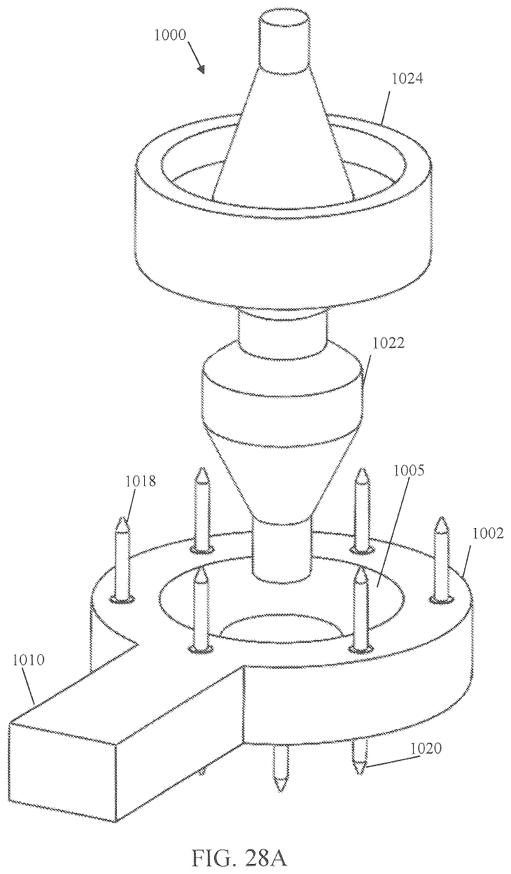

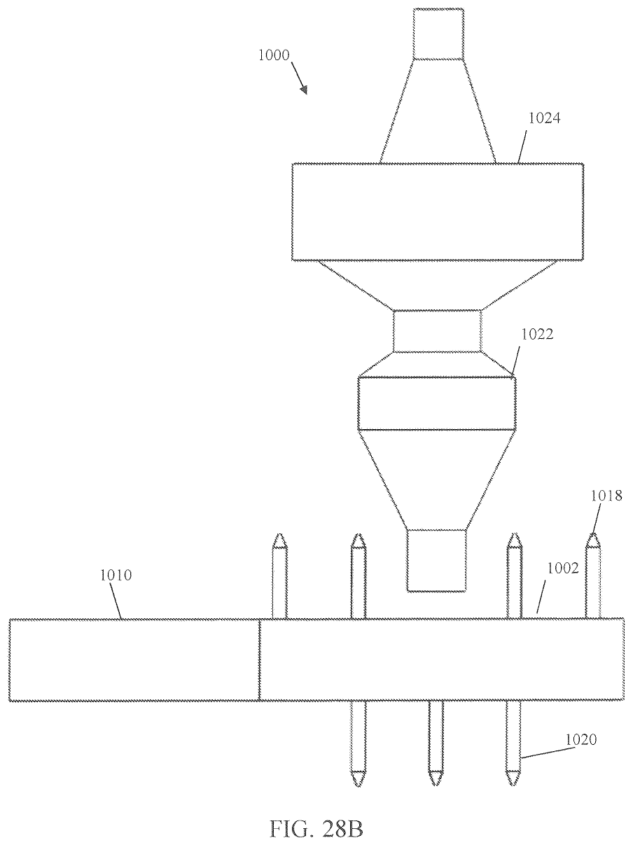

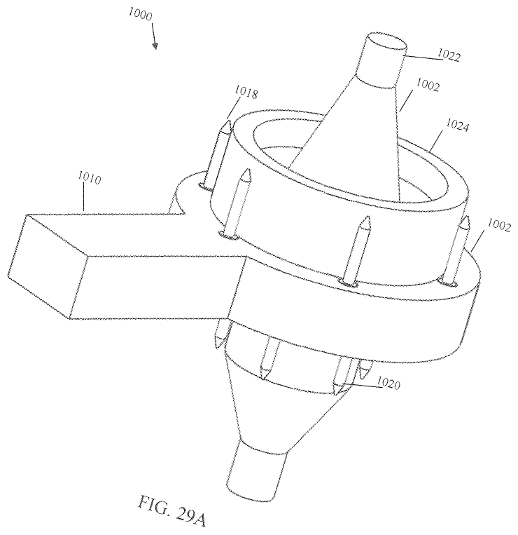

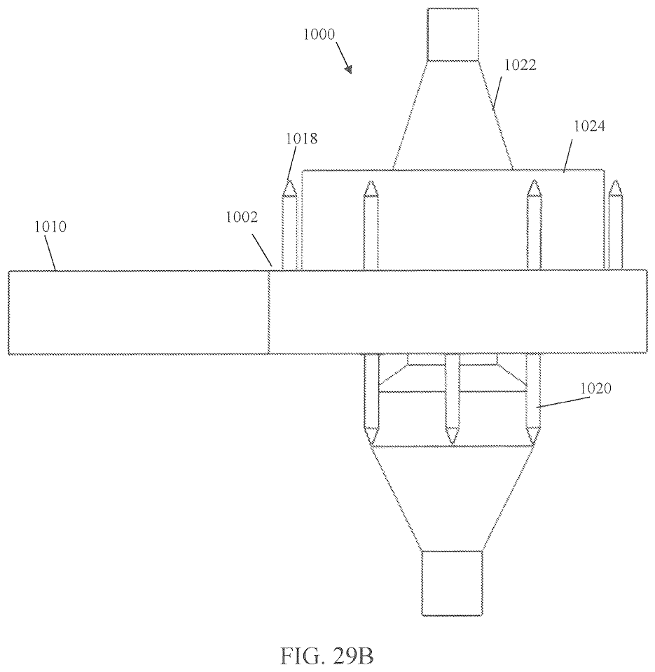

FIG. 28A-29B illustrate a holder 1022 for joining a cap element 1024 to a first coupling element 1002. A surgeon may join the cap element 1024 to the holder 1022 during a surgical procedure or the holder 1022 may be attached to a cap element 1024 prior to the procedure. The holder 1022 may be sized to be inserted into an aperture 1006 of the first coupling element 1022. For example, an end of the holder 1022 opposite the cap element 1024 may be inserted into the aperture 1024 such that the cap element 1024 is adjacent to a side of the first coupling element 1022 containing the first tissue grasping elements 1018. The holder 1022 may then be pulled through the aperture 1006, leaving the cap element 1024 in place secured to the first coupling element 1022.

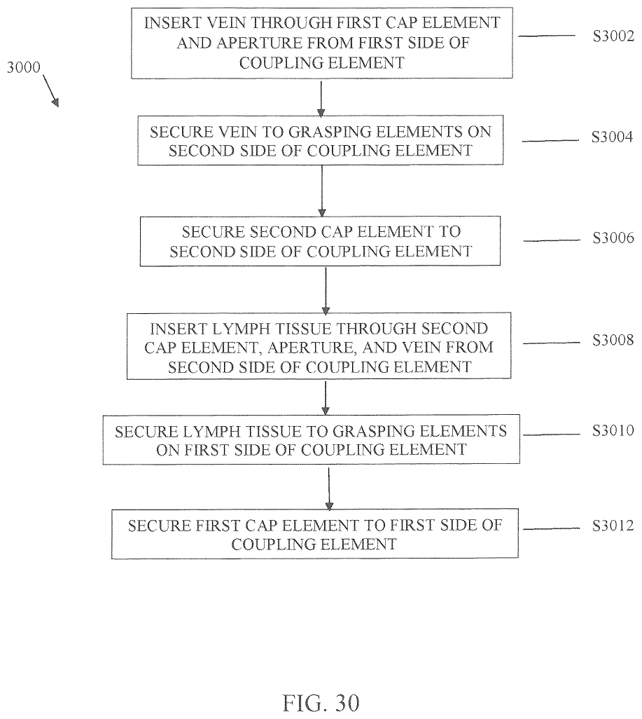

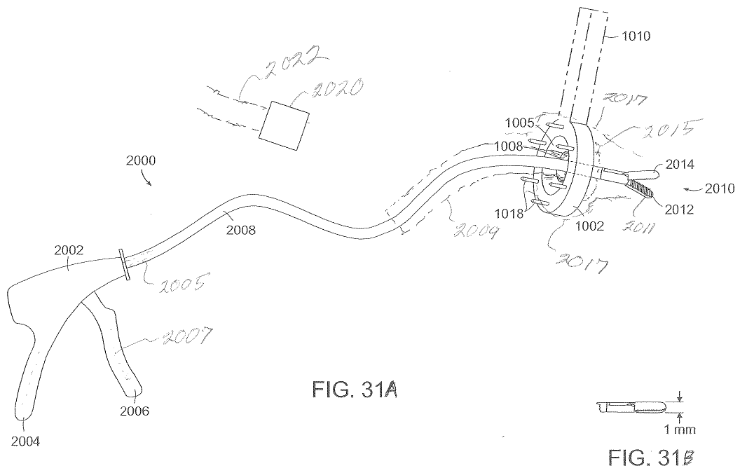

FIG. 30 depicts an exemplary method 3000 of performing a lymphovenous bypass surgical procedure. The method begins at S3002 when a vein or artery is inserted through a first cap element and the conical aperture of a first coupling element. The vein or artery is inserted from a first side of the fist coupling element where the conical aperture is widest. At S3004, the vein or artery is secured to grasping elements on a second side of the first coupling element. The vein or artery may be secured such that the vein or artery is widened to allow insertion of lymph channels. At S3006, a second cap element is secured to the second side of the first coupling element. In some embodiments, the second cap element is secured to the grasping elements on the second side.

At S3008, lymph tissue, for example lymph channels and associated fatty tissue, is inserted through the second cap element, the secured vein or artery, and the aperture of the first coupling element. At S3010, the lymph tissue is secured to grasping elements on a first side of the first coupling element. Then at S3012 the first cap element is secured to the first side of the first coupling element. In some embodiments, the first cap element is secured to the grasping elements on the first side.

Note that in certain embodiments the cap for the first coupling element can comprise a first cap element flexibly connected to a second cap element so as align the respective cap elements to the respective tissue grasping elements to simplify the process. In the present embodiments the caps engage one or more of the tissue grasping elements on each side. In other embodiments the caps can be coupled to features on the ring or the cap elements can connect to each other so as to enclose the tissue grasping elements. The caps can also comprise sidewall openings so as to avoid the need to thread the vein or artery through one cap element, and/or a sidewall opening to provide for the insertion of the lymphatic channels and/or the fatty tissue in which they are positioned into the second cap element into position for attaching to the first coupling element. A small forceps device such as the SpyBite™ biopsy forceps manufactured by Boston Scientific Corporation, Marlborough, MA, can be used to grasp tissue in the procedures described herein. A further improved forceps design better suited for this procedure has a working length of less than 12 cm with a cable diameter in the range of 1-1.5 mm or less and a jaw outer diameter in a range of less than 1-1.5 mm. The forceps are scissor actuated to simplify the grasping and manipulation of tissue as described herein to maneuver within the confined surgical space and grasp and manipulate objects during the procedure.

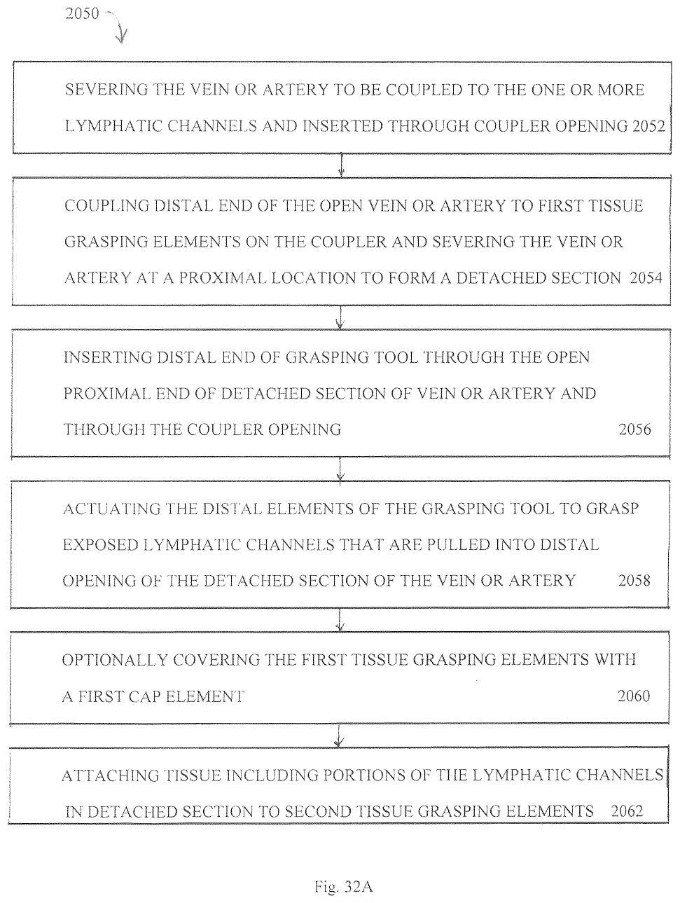

A further exemplary embodiment is illustrated in FIG. 31A that depicts a grasping tool 2000 having the dimensions to be directed through the conical opening 1008 on a first side of the coupling device 1002. The tool 2000 has a handle 2002 coupled to a proximal end of a tubular body 2008 that has one or more moveable tool elements 2010 at a distal end of the tubular body suitable to grasp one or more lymphatic channels. The distal tool elements 2012, 2014 pivot relative to a hinge to open and close (see FIG. 31B) in response to a manual operation of a handle actuator configured to be operated by a user. The tool elements can include a flexible material on the tissue contacting surfaces that compresses under grasping pressure applied by the user so as not to damage the lymphatic channels by excessive manual pressure. The outer surfaces of the distal tool elements are rounded smooth surfaces to slide through the detached section of vein or artery or graft 2009 during entry and removal. The outer diameter of the tube and the tool elements in the closed position is preferably between 0.5 and 1.5 mm. The length of the tubular portion of the device with the tool elements in the closed position is preferably between 10-25 cm, although lengths up to 40 cm can be used depending on the length of the detached artery section as described herein. The handle actuator can comprise a pistol grip with one or more moveable handle elements 2004, 2006 such that a user's hand can squeeze the handle elements towards each other to cause the grasping elements to move towards each other from the open position. The handle elements can have a textured surface 2011 with flexible material to provide tactile feedback to the user to avoid applying too much pressure and thereby risk damaging the tissue being grasped. The actuator can be spring loaded so that the distal tool elements are open unless locked in a closed position or by manual actuation with the handle actuator. The handle actuator can operate using cables 2005 extending through tube 2008 to the distal end to rotate the elements 2012, 2014 and also include handle elements 2004, 2006 with a textured surface 2007 with flexible surface elements such that a user's hand can sense a level of pressure being applied to the tissue being grasped by the tool and thereby prevent damaging the fragile lymphatic channel tissue. A vascular coupler 2020 can be used to connect the proximal open end of graft 2009 to an intact vein or artery 2022 and thereby restore flow that now comprises lymphatic fluid flowing through the coupler device, graft 2009 and into vein or artery 2022. Note that a cap 2015 can be applied to cover the ends of tissue grasping elements that retain the end of the vein or artery in place. The supporting tissue 2017, that partially includes the lymphatic channels that have been pulled into the graft 2009, is then pulled at least partially around the perimeter of the coupler to engage tissue grasping elements 1018.

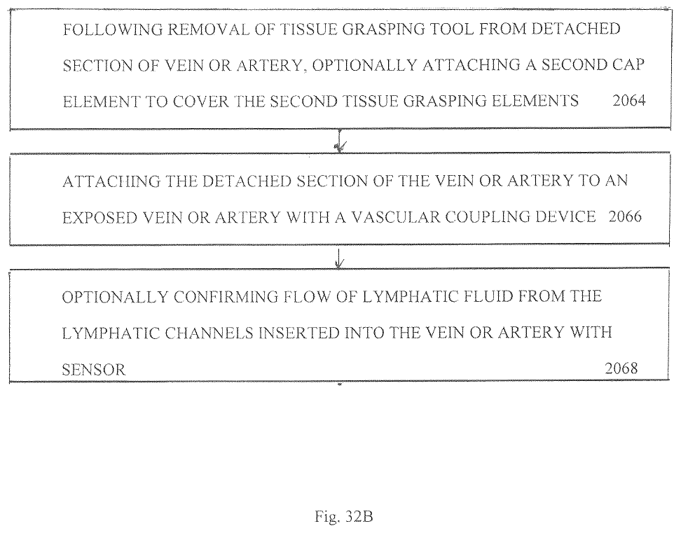

In a preferred method 2050 of inserting the lymphatic channels into the vein or artery is illustrated in FIGS. 32A and FIG. 32B. The vein or artery is severed 2052 and the end of the vein or artery is inserted through the coupler opening and attached 2054 to the tissue grasping elements on a first side of the unitary coupler. Alternatively, the graft can be severed at both ends and the grasping tool can be inserted through the graft to assist in holding the graft in place while it is inserted through the opening of the coupled device and attached to one side with the tissue grasping elements. As seen in FIGS. 32A and 32B the attached portion of the vein or artery is then severed at a proximal location after coupling to the coupler device to provide the graft or detached section of the vein or artery that will later be re-attached to the same end from intact vein from which it was severed. The detached section of the vein or artery has a proximal opening through which the distal end of the grasping tool is inserted 2056 to a position that extends through the opening of the unitary coupler and completely through the detached section of the vein or artery as shown in FIG. 31A. The distal elements 2012, 2014 of the grasping tool are actuated to grasp the exposed lymphatic channels which are then pulled 2058 into the distal end of the detached portion of the vein or artery, referred to herein as the graft, to a selected depth with the grasping tool. The exposed ends of the tissue grasping elements on the first side of the unitary coupler can optionally be covered 2060 by a first cap element as described herein. The tissue in which a portion of the lymphatic channels are situated is then pulled 2062 over the outer perimeter of the unitary coupler and attached to the tissue grasping elements on the second side of the unitary coupler. Note that the grasping tool has been removed from the coupler opening and the detached portion of the vein or artery so that the proximal opening of the vein or artery can then be re-attached with a vascular coupler. First, a second cap element can optionally be attached 2064 to the exposed ends of the tissue grasping elements on the second side of the unitary coupler. This step involves inserting the proximal end of the detached portion of the vein or artery through a second cap element opening.

An exposed end of an intact vein or artery is then attached (or re-attached) 2066 to the detached section of the vein or artery to re-establish blood flow within the formerly detached portion of the vein or artery that is now receiving lymph fluid through the lymphatic channels that have been inserted into the formerly detached portion of the vein or artery. Note that commercially available vascular coupler for connecting exposed ends of veins or arteries can be used for coupling the detached portion of the vein or artery to an exposed end of a vein or artery that are of substantially the same diameter. As previously described, the flow of lymphatic fluid into the vein or artery can be confirming by sensing 2068 fluid flow as previously described.

It will be appreciated by those skilled in the art that modifications to, and variations of the above described device and methods can be made without departing from the inventive concepts disclosed herein. Accordingly, the disclosure should not be viewed as limited except as by the scope and spirit of the appended claims.

1. A device for lymphovenous bypass surgery comprising:
a coupling device that couples one or more lymphatic channels into an open end of a vein or artery from supporting tissue, the coupling device having a first tissue grasping element that extends in a first direction from a first side and configured to attach to the vein or artery, and a second tissue grasping element that extends in a second direction from a second side and configured to attach to a portion of the supporting tissue; and
wherein the coupling device has an opening configured to receive the vein or artery.

2. The device of claim 1, wherein the first tissue grasping element extends from a coupling device surface on a first side of a plane extending through the coupling device and the second tissue grasping element extends from a further coupling device surface on an opposite side of the plane.

3. The device of claim 1, wherein the coupling element has a cone-shaped surface extending around an aperture of the coupling element along a central axis orthogonal to a plane extending between opposite sides of the coupling device.

4. The device of claim 1, wherein the first tissue grasping element and the second tissue grasping element each include one or more pins.

5. The device of claim 1, further comprising a first cap element that encloses the first tissue grasping element and a second cap element that encloses the second tissue grasping element.

6. (canceled)

7. The device of claim 5, wherein the first cap element is connected to the second cap element.

8. The device of claim 1, wherein the coupling device comprises a ring having a first radius defining positions of a plurality of first tissue grasping elements spaced around a first circumference.

9. The device of claim 8, wherein the ring has a second radius defining positions of a plurality of second tissue grasping elements spaced around a second circumference.

10. The device of claim 9, wherein the second radius is smaller than the first radius.

11. (canceled)

12. (canceled)

13. The device of claim 1, wherein the first and second tissue grasping elements are equally spaced around respective opposing sides of the coupling device.

14. The device of claim 1, wherein the coupling device has an opening with a diameter in a range of 0.5-1.5 mm.

15. The device of claim 1, further comprising a grasping tool including a tubular body that is configured to extend through an opening in the coupling element.

16. The device of claim 15, wherein the tubular body having has a diameter in a range of 0.5-1.5 mm.

17. The device of claim 15, wherein the grasping tool has one or more distal elements configured to grasp the one or more lymphatic channels.

18. The device of claim 17, wherein the grasping tool further comprises an actuator that moves the one or more distal elements on the tubular body to grasp tissue.

19. (canceled)

20. (canceled)

21. The device of claim 15, wherein the tubular body extends through the coupling element to grasp the one or more lymphatic channels with distal elements and pull ends of the one or more lymphatic channels through the opening of the coupling element and into the vein or artery.

22. The device of claim 15, wherein the coupling device has an outer circumference configured to have a size such that the supporting tissue is positioned around at least a portion of the circumference to engage the second tissue grasping element.

23. The device of claim 15, wherein the grasping tool includes one or more manually actuated elements coupled to distal grasping elements, the manually actuated elements having flexible surface elements such that a user adjusts an applied pressure to tissue being grasped using tactile sensing of applied pressure.

24. (canceled)

25. (canceled)

26. A method of performing a lymphovenous bypass surgical procedure comprising:
attaching a coupling element having first tissue grasping elements on a first side of the coupling element to a vein or artery of the patient;
inserting one or more lymphatic channels into an open end of the vein or artery to deliver lymph fluid from the one or more lymphatic channels into the vein or artery;
attaching second tissue grasping elements on a second side of the coupling element to tissue including a portion of the one or more lymphatic channels, wherein the second tissue grasping elements extend from the second side second side of the coupling element.

27. (canceled)

28. The method of claim 26, wherein positioning the at least one lymphatic channel into an open end of the vein or artery causes the at least one lymphatic channel to intussuscept the vein by extending a distance into the vein.

29. The method of claim 26, wherein one or more of of the one or more lymphatic channels extend to a depth of at least 1 mm inside the vein.

30. The method of claim 26, wherein one or more of of the one or more lymphatic channels are at least partially separated from supporting tissue for insertion into the vein or artery and wherein a separated portion of the supporting tissue extends around a perimeter of the coupling device.

31. (canceled)

32. The method of claim 26, further comprising operating a grasping tool that grasps the one or more lymphatic channels for insertion into the vein or artery.

33. The method of claim 32, wherein the grasping tool includes a tubular body and further comprising inserting the tubular body through an opening in the coupling device to grasp the one or more lymphatic channels.

34. The method of claim 33, wherein a distal end of the tubular body comprises one or more moveable distal elements configured to grasp tissue and further comprising actuating the one or more moveable distal elements to grasp and position the lymphatics channels within the vein or artery.

35. (canceled)

36. The method of claim 34, wherein the supporting tissue is positioned at least partially around a perimeter of the coupler device and attached to tissue grasping elements.

37. The method of claim 36, further comprising positioning a cap over the tissue grasping elements coupled to the supporting tissue.

38. The method of claim 36, further comprising coupling a proximal end of the detached section of the vein or artery to an intact vein or artery with a vascular coupler to establish a flow of lymph fluid from the lymphatic channels through the coupling device opening and into the intact vein or artery.

39. (canceled)

40. (canceled)
